(19) United States
(12) Patent Application Publication
(10) Pub. No.: US 2009/0305437 A1
(43) Pub. Date: Dec. 10, 2009

(54) FABRICATION OF INORGANIC MATERIALS USING TEMPLATES WITH LABILE LINKAGE

(75) Inventors: Pierre-Marc Allemand, San Jose, CA (US); Manfred Heidecker, Mountain View, CA (US); Gregory L. Kirk, Pleasanton, CA (US); Xina Quan, Saratoga, CA (US); Cheng-I Wang, Foster City, CA (US)

Correspondence Address: SEED INTELLECTUAL PROPERTY LAW GROUP PLLC, 701 FIFTH AVE, SUITE 5400, SEATTLE, WA, 98104, US

(73) Assignee: CAMBRIOS TECHNOLOGIES CORPORATION, Sunnyvale, CA (US)

(21) Appl. No.: 12/282,160

(22) PCT Filed: Mar. 8, 2007
(86) PCT No.: PCT/US2007/005998

§ 371 Date: Jun. 1, 2009

Related U.S. Application Data
(60) Provisional application No. 60/780,783, filed on Mar. 9, 2006.

(51) Int. Cl. H01L 21/36; C07K 2/00; C07K 16/00

(52) U.S. Cl. 438/1; 530/300; 530/391.1; 257/E21.461

(57) ABSTRACT

A method of forming an integrated circuit layer material is described, comprising depositing a layer of templates on a substrate, said template including a first binding site having an affinity for the substrate, a second binding site having an affinity for a target integrated circuit material and a protecting material coupled to the second binding site via a labile linkage to prevent the binding site from binding to the target integrated circuit material; exposing the template to an external stimulus to degrade the labile linkage; removing the protecting material; and binding the integrated circuit material to the second binding site.

### CROSS-REFERENCE TO RELATED APPLICATION

This application claims the benefit under 35 U.S.C. § 119(e) of U.S. Provisional Patent Application No. 60/780,783, filed Mar. 9, 2006, where this provisional application is incorporated herein by reference in its entirety.

### BACKGROUND OF THE INVENTION

1. Field of the Invention

This invention relates to templates used to fabricate inorganic materials, in particular embodiments, inorganic materials suitable as integrated circuit components, and methods of fabricating the same.

2. Description of the Related Art

As traditional “top down” lithographic fabrication techniques rapidly approach their physical limits in the ability to produce sub-50 nm features, “bottom up” methods characterized with directed self-assembly of functionalities make it feasible to fabricate nanostructured devices. These nanostructure devices comprising molecule-sized building blocks promise to open up numerous novel applications in quantum computing, sensing, flexible electronics and integration with biotechnology, in addition to high-speed, high-device-density microprocessors.

Typically, as electronic components become miniaturized, the fabrication and manipulation of these building blocks become difficult and unreliable. In contrast, organic molecules in biological systems exhibit a remarkable control over nucleation and mineralization of inorganic materials, as well as over the assembly of crystallites and other nanoscale building blocks into complex structures required for biological functions.

The feasibility of “bottom-up” nanoscale fabrication based on directed self-assembly is therefore largely inspired by nature. Based on their ability to direct the assembly of inorganic material into controlled and sophisticated structures, biomolecules are exploited as templates to produce functional nanostructures. Intense research efforts have been devoted to identifying biomolecule templates and developing assembly methods that mimic or exploit the recognition capabilities and interactions found in biological systems.

In particular, biomolecules having sequence specific affinities to certain inorganic materials form the basis of a functional hybrid system in which organics and inorganics interface in an orderly and controlled manner. Advantageously, well-established techniques and protocols in molecular biology, such as nucleic acid-based design, can be carried over to this new approach of material engineering using biomolecules as templates. For example, proteins or peptides with specific binding recognition for certain substrate or functional entities (e.g., nanoparticles) can be selected from a massively diverse library and amplified. The amino acid sequence of the protein or peptide can be rapidly determined. This ability to design protein templates based on genetics therefore ensures total control over the molecular structure of the protein templates. See, e.g., Mao, C. B. et al., Virus-Based Toolkit for the Directed Synthesis of Magnetic and Semiconducting Nanowires,” (2004) Science, 303, 213-217; Belcher, A. et al., “Ordering of Quantum Dots Using Genetically Engineered Viruses,” (2002) Science 296, 892-895; Belcher, A. et al., “Selection of Peptides with Semiconductor Binding Specificity for Directed Nanocrystal Assembly,” (2000) Nature 405 (6787) 665-668. Furthermore, Reiss et al., “Biological Routes to Metal Alloy Ferromagnetic Nanostructures” (2004) Nanoletters, Vol. 4, No. 6, 1127-1132, describes peptides for binding to metals, including mediating nanoparticle synthesis. Flynn, Mao, et al., “Synthesis and Organization of Nanoscale II-VI semiconductor materials using evolved peptide specificity and viral capsid assembly,” (2003) J. Mater. Sci., 13, 2414-2421, describes peptides for binding to and nucleation of semiconductor nanoparticles. Mao, C. B. et al., “Viral Assembly of Oriented Quantum Dot Nanowires,” (2003) PNAS, vol. 100, no. 12, 6946-6951, further describes peptides for binding to and nucleation of semiconductor nanoparticles. All of the above references are hereby incorporated by reference in their entireties.

Nucleic acid-based and polypeptide-based design of biomolecule templates, while powerful, can be inflexible once a binding site has been established based on a specific sequence. Accordingly, there remains a need in the art to be able to modify biomolecules by altering the accessibility of a binding site, such as a binding site for an inorganic material, e.g., to mediate the interface between the organics and inorganics.

### BRIEF SUMMARY OF THE INVENTION

In one embodiment, the present invention provides a method of forming an integrated circuit layer material comprising: depositing a layer of templates on a substrate, said template including a first binding site having an affinity for the substrate, a second binding site having an affinity for a target integrated circuit material and a protecting material coupled to the second binding site via a labile linkage to prevent the binding site from binding to the target integrated circuit material; exposing the template to an external stimulus to degrade the labile linkage; removing the protecting material; and binding the integrated circuit material to the second binding site.

In another embodiment, the present invention provides a method of forming a target inorganic material comprising: depositing a biomolecular template on a substrate, the biomolecular template having a multifunctional biomolecule including a first binding site coupled to the substrate and a second binding site having an affinity for the target inorganic material, and a protecting group coupled to the multifunctional biomolecule via a labile linkage to prevent the second binding site from binding to the target inorganic material; exposing the biomolecular template to an external stimulus to degrade the labile linkage; removing the protecting group; and binding the target inorganic material to the second binding site.

In another embodiment, the present invention describes a method of patterning a target inorganic material on a substrate. The method comprises: depositing a plurality of biomolecular templates on the substrate to form a template layer, each biomolecular template having a multifunctional biomolecule including a first binding site coupled to the substrate and a second binding site having an affinity for the target inorganic material, and a protecting group coupled to the multifunctional biomolecule via a labile linkage such that the second binding site is prevented from binding to the target inorganic material; exposing, according to a selected pattern, a region of the template layer to an external stimulus; deprotecting the second binding sites of the biomolecular template in the region subjected to the external stimulus by degrading the labile linkages thereof; and binding the target inorganic material to the second binding sites in the region.

In one embodiment, the present invention provides a biomolecular template suitable for directing the assembly of an inorganic material, such as inorganic nanoparticles. The binding behavior of the biomolecular template with respect to the inorganic material is mediated by a labile protecting group as part of the template. More specifically, the labile protecting group blocks the access to a binding site having an affinity for the inorganic material, but can be removed to allow for access to the binding site in a controlled manner.

According to this embodiment, the biomolecular template comprises a multifunctional biomolecule including a first binding site having an affinity for a substrate and a second binding site having an affinity for a target inorganic material; and a protecting group coupled to the multifunctional biomolecule via a labile linkage, the protecting group preventing the second binding site from binding to the target inorganic material.

In a further embodiment, the present invention provides a biomolecular conjugate suitable for nanostructure fabrication. The biomolecular conjugate comprises a multifunctional biomolecule including a first binding site having an affinity for a substrate and a second binding site coupled to the multifunctional biomolecule via a labile linkage; and a target inorganic material conjugated to the second binding site.

In another embodiment, a method of patterning a target inorganic material layer composed of a plurality of nanoparticles is described. The method comprises: depositing a plurality of biomolecular conjugates on a substrate, each said biomolecular conjugate including a multifunctional biomolecule having a first binding site coupled to the substrate and a second binding site conjugated to the nanoparticle, the second binding site being coupled to the multifunctional biomolecule via a labile linkage; exposing, according to a selected pattern, a region of the biomolecular conjugates to an external stimulus; and detaching the nanoparticles from the biomolecular conjugates in the region subjected to the external stimulus.

In a further embodiment, a method of patterned formation of a target inorganic material layer is described. The method comprises: depositing a layer of multifunctional biomolecules on a substrate, each multifunctional biomolecule including a first binding site coupled to the substrate, a labile linkage and a second binding site having an affinity for a target inorganic material, exposing, according to a selected pattern, a region of the layer of the multifunctional biomolecules to an external stimulus; removing the second binding sites from the multifunctional biomolecules in said region by cleaving the labile linkages thereof; and contacting the substrate to the target inorganic material whereby the target inorganic material binds to the second binding sites of the multifunctional biomolecules in a region not exposed to the external stimulus.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIGS. 1A-1B illustrate schematically an embodiment of the present invention in which a biomolecule can be manipulated to bind to a target material;

FIGS. 1C-1D illustrate schematically an embodiment of the present invention in which a biomolecule conjugated to a target material can be removed upon exposure to an external stimulus;

FIG. 1E illustrates schematically an embodiment of the present invention in which a multifunctional biomolecule presents four binding sites.

FIG. 2A is a schematic illustration of a biomolecular template having a labile protecting group;

FIG. 2B shows a biomolecular template undergoes deprotection and nucleation process;

FIGS. 3A-3C show a light-triggered formation of an inorganic material layer according to a selected pattern;

FIGS. 4A-4D show a heat-triggered formation of an inorganic material layer according to a selected pattern;

FIG. 5 shows a formation of an inorganic material layer on a seed layer;

FIGS. 6A-6B shows a formation of an inorganic material layer according to a selected pattern without using a mask;

FIGS. 7A-7B show deposition processes of biomolecular templates on a substrate by printing;

FIG. 8 illustrates a biomolecular conjugate having a labile linkage;

FIG. 9 illustrates a biomolecular conjugate from which an inorganic nanoparticle is removed through a cleavage of a binding site;

FIG. 10 illustrates a biomolecular conjugate from which an inorganic nanoparticle is removed through disruption of a binding site;

FIGS. 11A-11C illustrate schematically etching of an inorganic material layer according to a selected pattern; and

FIGS. 12A-12C illustrate schematically the formation of an inorganic material layer according to a selected pattern.

FIG. 13 illustrate schematically the patterning an inorganic material layer using a seed material.

### DETAILED DESCRIPTION OF THE INVENTION

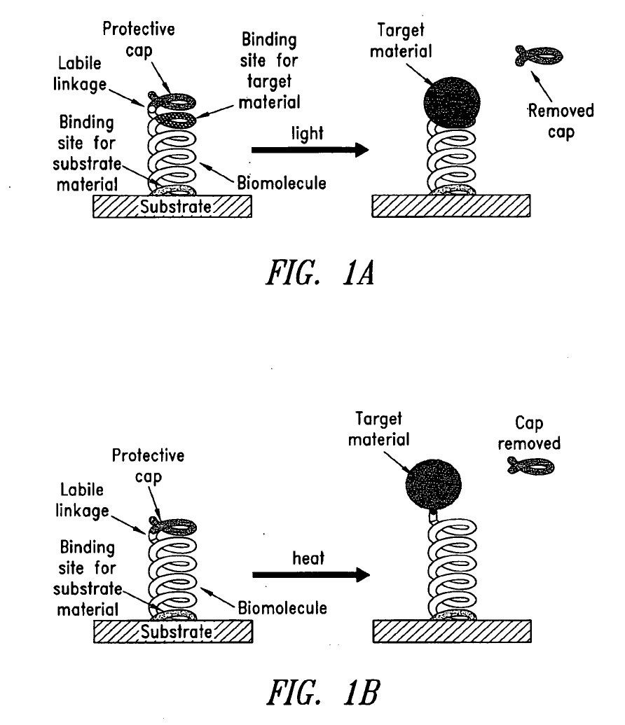

FIGS. 1A-1B illustrate schematically an embodiment of the present invention. FIG. 1A shows a template, e.g., a multifunctional biomolecule having a first binding site coupled to a substrate material and a second binding site blocked by a protective group, the protective group being coupled to the multifunctional biomolecule via a labile linkage. The labile linkage is a chemical moiety, including a covalent bond, which degrades upon exposure to an external stimulus.

In FIG. 1A, the labile linkage is photo-labile hence degradable upon irradiation with light. Therefore, when exposed to light, the protective cap is removed and the second binding site becomes accessible to binding to a target material. FIG. 1B illustrates a similar process as in FIG. 1A, except that the labile linkage is thermal-labile, hence degradable upon exposure to heat. As shown, the labile linkage can be manipulated by external stimuli to control the binding activity and accessibility of the second binding site of the biomolecule. In addition to light and heat as illustrated, enzymes and chemical reagents or combinations thereof can also be used to mediate the accessibility of the second binding site.

Biomolecules such as those illustrated in FIGS. 1A and 1B can be used as templates to direct the deposition and patterning of a target material layer on a substrate. The presence of the labile linkage in the biomolecular template affords a means to manipulate the binding behavior of the biomolecular template. For example, once the biomolecular templates are deposited on a substrate, the binding sites previously blocked or capped can be freshly exposed or created upon cleavage of the labile linkage. By manipulating the labile linkages of the biomolecular templates in desired regions, a target material layer can be formed according to a selected pattern.

FIGS. 1C and 1D illustrate schematically a further embodiment of the present invention. FIG. 1C shows a multifunctional biomolecule bound to a substrate by a first binding site. The multifunctional biomolecule further comprises a second binding site and a labile linkage. The multifunctional biomolecule is conjugated, via its second binding site, to a target material. The labile linkage can be controlled to cleave upon exposure to an external stimulus. The cleavage causes the removal of the second binding site and the target material conjugated thereto.

The controlled removal of a binding site can be applied to patterning a target inorganic material layer, as shown in FIG. 1D. A plurality of multifunctional biomolecules are formed on a substrate. The substrate can be subjected to an external stimulus according to a selected pattern. In the region exposed to the external stimulus, the second binding sites are removed and no target material is formed. In the region not exposed to the external stimulus, the second bind sites bind to the target material to create the pattern as illustrated.

The templates of the present invention are not limited to biomolecules having two binding sites. Multifunctional biomolecules having more than two binding sites are suitable as versatile templates for creating complex patterns through external manipulation of their binding activities. FIG. 1E illustrates schematically an embodiment of the present invention in which a multifunctional biomolecule presents four binding sites. More specifically, the multifunctional biomolecule comprises a first binding site bound to a first inorganic material, a second binding site having an affinity for a second inorganic material, a third binding site having an affinity for a third inorganic material, and a fourth binding site having an affinity for a substrate. The first, second and third inorganic material can be the same or different. Preferably, each binding site binds to a given inorganic material with specificity, as defined herein. More over, each binding site is connected to the body of the multifunctional biomolecule via a first, second and third labile linkages, respectively.

The binding activity of each binding site can be manipulated through the cleavage of the labile linkage to which the binding site is connected. Preferably, each labile linkage is selectively cleavable by a different external stimulus. External stimuli can be different if they belong to different categories, e.g., light irradiation and enzymatic treatment. External stimuli of the same category can also be differentiated by their particular attributes. For example, different labile linkages can be susceptible to cleavage upon light irradiation of different wavelengths, or upon treatment of different enzymes. Accordingly, the multiple labile linkages of the multifunctional biomolecule can be selectively cleaved according to a design, to expose a binding site for binding or to remove a binding site.

The versatility of the binding activities of a multifunctional biomolecule is further illustrated in FIG. 1E. For example, the cleavage of the first labile linkage causes the removal of the first inorganic material. The cleavage of the second labile linkage exposes the second binding site for binding to the second inorganic material, and so forth. Depends on the particular need, an end user can access a desired binding site by choosing the appropriate labile linkage to cleave. Moreover, by using a plurality of the multifunctional biomolecules as the templates, complex patterns of inorganic materials can be formed by serial manipulation of the external stimuli.

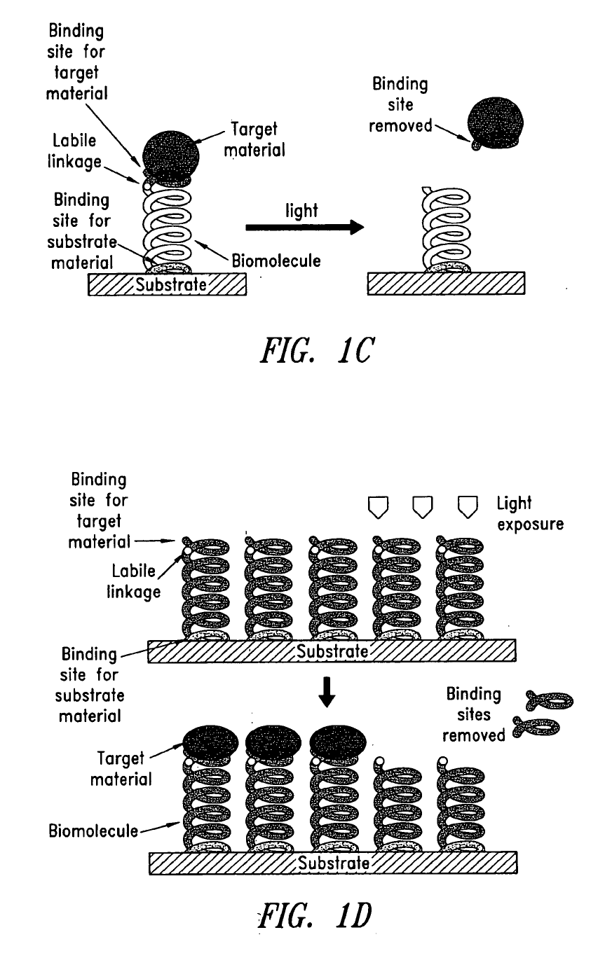

FIG. 2A illustrates in detail one embodiment of the present invention. A biomolecular template 2 suitable to direct the assembly of an inorganic material on a substrate 4. The biomolecular template 2 comprises a multifunctional biomolecule 8 including a first binding site 12 having an affinity for the substrate 4, a second binding site 14 having an affinity for a target inorganic material (not shown), and a protecting group 16 coupled to the multifunctional biomolecule 8 via a labile linkage 18. The protecting group 16, also referred herein as “labile protecting group”, serves to block the second binding site 14 from binding to the inorganic material and can be removed to re-expose the second binding site 14.

FIG. 2A therefore demonstrates schematically the biomolecular template 2 in which the second binding site 14 is protected by the labile protecting group 16 and is inaccessible for binding. In response to an external stimulus such as light, heat, enzyme or chemical reagent, the labile linkage 18 can be cleaved and the labile protecting group 16 removed, thereby activating the second binding site 14 for binding thereto.

The components of a biomolecular template are now described in more detail below.

### 1. Multifunctional Biomolecules:

“Multifunctional biomolecule” refers to a biomolecule having at least two functionalities, which correspond to a binding site having an affinity for a substrate and at least another binding site having an affinity for a target inorganic material. Multifunctional biomolecule includes “bifunctional biomolecule” having two functionalities, and “trifunctional biomolecule” having three functionalities, and so forth. As noted above, multifunctional biomolecules may further comprise multiple labile linkages, which will be discussed in more detail below.

“Biomolecule” refers to a carbon-based organic molecule of a biological origin. Typically, a biomolecule comprises a plurality of subunits (building blocks) joined together in a sequence via chemical bonds. Each subunit comprises at least two reactive groups such as hydroxyl, carboxylic and amino groups, which enable the bond formations that interconnect the subunits. Examples of the subunits include, but not limited to: amino acids (both natural and synthetic) and nucleotides. Examples of biomolecules include peptides, proteins (including cytokines, growth factors, etc.), nucleic acids, polynucleotides, viruses, cells, cofactors, tissues, organs, fatty acids, sugars, organic polymers and other simple or complex carbon-containing molecules, and combinations thereof.

The biomolecules of the present invention are characterized by their ability to recognize and bind to an inorganic material with specificity and/or selectivity. In particular, biomolecules comprising subunits of amino acids are found to exhibit sequence-specific binding behavior toward inorganic materials. Examples of amino acid-based biomolecules include, but are not limited to peptides, antibodies, block copolypeptides or amphiphilic lipopeptides.

As used herein, “peptide” refers to a sequence of two or more amino acids joined by peptide (amide) bonds, including proteins. The amino-acid building blocks (subunits) include naturally-occurring α-amino acids and/or unnatural amino acids, such as β-amino acids and homoamino acids. Moreover, an unnatural amino acid can be a chemically modified form of a natural amino acid. In particular, an amino acid coupled to a labile protecting group can be incorporated into a peptide sequence.

As used herein, “block copolypeptide” refers to polypeptides having at least two covalently linked domains (“blocks”), one block having amino acid residues that differ in composition from the composition of amino acid residues of another block. “Amphiphilic lipopeptide” refers to a hydrophilic peptide head group conjugated to a hydrophobic group, such as a fatty acid or steroid.

As used herein, “polynucleotide” refers to an oligomer of about 3-50 nucleotide units interconnected by a phosphate backbone. A polynucleotide of 2-10 nucleotide units is also referred to as “oligonucleotide”. The nucleotide subunits include all major heterocyclic bases naturally found in nucleic acids (uracil, cytosine, thymine, adenine and guanine) as well as naturally occurring and synthetic modifications and analogs of these bases such as hypoxanthine, 2-aminoadenine, 2-thiouracil and 2-thiothymine. The nucleotide subunits further include deoxyribose, ribose and modified glycosides.

(a) Material-Specific Binding Activities

The multifunctional biomolecules of the present invention exhibit characteristic material-specific binding activities. These binding activities can be manipulated through a number of external stimuli, as will be discussed in detail below.

“Binding site”, used interchangeably herein with “binding sequence”, refers to the minimal structural elements within a biomolecule that are associated with or contribute to the biomolecule's binding activities. As used herein, the terms “bind” and “couple” and their respective nominal forms are used interchangeably to generally refer to one entity being attracted to another to form a stable complex.

The underlying force of the attraction, also referred herein as “affinity” or “binding affinity”, can be any stabilizing interaction between the two entities, including adsorption and adhesion. Typically, the interaction is non-covalent in nature; however, covalent bonding is also possible. A covalent bond is formed between two atoms sharing at least a pair of electrons. A non-covalent bond can be based on van de Waals force, electrostatic interaction, hydrogen bonding, dipole-dipole interaction or a combination thereof.

Typically, a binding site comprises a functional group of the biomolecule, such as thiol (—SH), hydroxy (—OH), amino (—NH₂) and carboxylic acid (—COOH). For example, the thiol group of a cysteine effectively binds to a gold particle (Au). More typically, a binding site is a sequence of subunits of the biomolecule and more than one functional groups may be responsible for the affinity. Additionally, conformation, secondary structure of the sequence and localized charge distribution can also contribute to the underlying force of the affinity.

The magnitude of the binding affinity can be quantitatively represented by an association constant of the binding equilibrium. Known methods in the art, such as Langmuir model for adsorption of analytes on a surface, can be used to measure the association constant. Typically, the association constant can be greater than 1×10⁵ M⁻¹, greater than 1×10⁷ M⁻¹, greater than 1×10⁹ M⁻¹ or greater than 1×10¹¹ M⁻¹.

The binding activities of the biomolecules of the present invention include but are not limited to: their ability to specifically recognize and bind to a material or to display a favorable affinity toward one material over another, also referred as “selective binding”. “Specifically” and “selectively” are terms of art that would be readily understood by the skilled artisan to mean, when referring to the binding capacity of a biomolecule, a binding reaction that is determinative of the presence of the substrate in a heterogeneous population of other substrates, whereas the other substrates are not bound in a statistically significant manner under the same conditions. Specificity can be determined using appropriate positive and negative controls and by routinely optimizing conditions. The phrase further applies to a binding reaction that is determinative of the presence of the target inorganic material in a heterogeneous population of other inorganic materials.

The terms “conjugate” and “conjugation” refer in general to a process in which a multifunctional biomolecule directly binds to a target inorganic material, as defined herein. For example, a multifunctional biomolecule can be conjugated to a pre-made nanoparticle much the same way as a ligand binding to a target. See, e.g., Reiss et al., Nanoletters (Supra).

The terms “nucleate” and “nucleation” refer to a process in which a precursor material is converted to a target inorganic material in the presence of a biomolecule. During the nucleation process, the in situ generated target inorganic material binds to and grows on the biomolecule. In one embodiment, the target inorganic material is a nanoparticle, as defined herein. For example, peptides of certain sequences selectively nucleate metal nanoparticles through reduction of a metal salt in a solution. Likewise, certain peptides selectively nucleate semiconductor nanoparticles. See, e.g., Flynn, Mao, et al., (2003) J. Mater. Sci. (supra); Mao, Flynn et al., (2003) PNAS (supra).

In a further embodiment, the initially nucleated nanoparticle can act as a seed material that catalyzes the growth of another target inorganic material. The term “seed material” therefore refers to a first inorganic material that causes the growth of a second inorganic material thereon. The first and second inorganic material may be the same or different. For example, when a seed material is exposed to a precursor of a second target inorganic material in a solution phase, the seed material catalyzes the conversion of the precursor into the second target inorganic material. Typically, the second target inorganic material can form a “shell” to the “core” represented by the seed material. More typically, the second target inorganic material forms a continuous layer over a seed material layer. This process is also referred to as “mineralization”. More particularly, when the second target inorganic material is a metal, the process forming a metal layer over a seed layer is also referred to as “metallization” or “plating”.

In another embodiment, the templates are deposited in such an ordered way as to induce the nucleation of nanoparticles with a preferred orientation or crystalline morphology. Individual templates can nucleate particular crystalline morphologies. When these templates are deposited with a particular orientation, packing, or spatial resolution due to the first binding site, the nanoparticles will be nucleated with similar or identical orientations. This can lead to the formation of highly ordered inorganic material, particularly after a thermal annealing step which fuses the nanoparticles together. A similar phenomenon is observed when mineralization occurs on peptide binding sequences fused onto biological scaffolds or particles, e.g. the pVIII coat proteins on M13 coliphage (see, e.g., Mao, C. B. et al., Virus-Based Toolkit for the Directed Synthesis of Magnetic and Semiconducting Nanowires,” (2004) Science, 303, 213-217).

Suitable multifunctional biomolecules are therefore selected based on such criteria as specific binding characteristics toward a given substrate, as well as toward one or more target inorganic material, collectively referred as “material” herein.

As used herein, a “substrate material” or “substrate” is a solid or semi-solid surface to which biomolecules attach through either covalent or non-covalent interactions. A substrate is typically an inorganic material, as defined herein. A substrate can also be organic, such as a polymer. In one embodiment, a substrate is a micro-fabricated material.

Examples of suitable substrate materials include, but are not limited to: a semiconductor material (e.g., silicon, germanium, etc.), Langmuir films, glass (including functionalized glass), ceramic, carbon, a polymer material, including polycarbonates, polyimides (e.g., Kaptone®), polystyrene, PTFE (e.g., Teflon®) and polyesters (e.g., Mylar®), a dielectric material, mica, quartz, gallium arsenide, metal, metal alloy, metal oxides, fabric, and combinations thereof. Typically, the substrate comprises functional groups such as amino, carboxyl, thiol or hydroxyl on its surface. The surface may be large or small and not necessarily uniform but should act as a contacting surface (not necessarily in monolayer). The substrate may be porous, planar or nonplanar. The substrate includes a contacting surface that may be the substrate itself or an additional layer. The additional layer, also referred herein as a “seeding layer”, will be described in more details in connection with deposition methods of the biomolecules on the substrate.

The term “inorganic material” refers to non-carbon based materials, including metals, metal oxides, metal alloys, semiconductive materials, minerals, ceramic, glass, salts, and combinations thereof. Metals may include Ag, Au, Sn, Zn, Ru, Pt, Pd, Cu, Co, Ni, Fe, Cr, W, Mo, Ba, Sr, Ti, Bi, Ta, Zr, Mn, Pb, La, Li, Na, K, Rb, Cs, Fr, Be, Mg, Ca, Nb, Tl, Hg, Rh, Sc, Y, or their alloys and oxides. Inorganic materials may also include, e.g., high dielectric constant materials (insulators) such as barium strontium titanate, barium zirconate titanate, lead zirconate titanate, lead lanthanum titanate, strontium titanate, barium titanate, barium magnesium fluoride, bismuth titanate, strontium bismuth tantalite, and strontium bismuth tantalite niobate, or variations, thereof, known to those of ordinary skill in the art.

Table 1 shows examples of peptides exhibiting specific affinity for a variety of inorganic materials.

TABLE 1

| | | |
|---|---|---|
| Peptide Sequence | Material | Type of Binding |

| | | |
|---|---|---|
| CNNPMHQNC | ZnS | nucleation, affinity^{1,2,3,4} |
| LRRSSEAHNSIV | ZnS | nucleation, affinity^{1,3,4} |
| QNPIHTH | PbS | conjugation¹ |
| CTYSRLHLC | CdS | nucleation, affinity¹ |
| SLTPLTTSHLRS | CdS | nucleation, affinity¹ |
| WDPYSHLLQHPQ | Streptavidin | conjugation⁵ |
| HNKHLPSTQPLA | FePt | nucleation, affinity^{6,7} |
| CNAGDHANC | CoPt | nucleation, affinity⁶ |
| SVSVGMKPSPRP | L10 FePt: | nucleation, affinity⁷ |
| VISNHRESSRPL | L10 FePt: | nucleation, affinity⁷ |
| KSLSRHDHIHHH | L10 FePt: | nucleation, affinity⁷ |
| VSGSSPDS | Au | nucleation, affinity⁸ |
| AEEEED | Ag, Co₃O₄ | nucleation, affinity⁹ |
| THRTSTLDYFVI | PPyCl | affinity¹⁰ |
| KTHEIHSPLLHK | CoPt | affinity |
| EPGHDAVP | Co²⁺ | nucleation, affinity¹¹ |
| HTHTNNDSPNQA | GaAs | affinity^{12,13} |
| DVHHHGRHGAEHADI | CdS | nucleation, affinity¹⁴ |
| KHKHWHW | ZnS, Au, CdS | affinity¹⁵ |
| RMRMKMK | Au | affinity¹⁵ |
| PHPHTHT | ZnS | affinity¹⁵ |
| CSYHRMATC | Ge dislocations | affinity¹⁶ |
| CTSPHTRAC | Ge dislocations | affinity¹⁶ |
| LKAHLPPSRLPS | Au | affinity⁹ |

¹Flynn, C. E. et al., “Synthesis and organization of nanoscale II-VI semiconductor materials using evolved peptide specfifcity and viral capsid assembly,” (2003) J. Mater. Sci., 13, 2414-2421.
²Lee, S-W et al., “Ordering of Quantum Dots Using Genetically Engineered Viruses,” (2002) Science 296, 892-895.
³Mao, C. B. et al., “Viral Assembly of Oriented Quantum Dot Nanowires,” (2003) PNAS, vol. 100, no. 12, 6946-6951.
⁴US2005/0164515
⁵Lee, S-W et al., “Viral-based alignment of inorganic, organic and biological nanosized materials” (2003) Advanced Material (Weinheim, Germany) 15(9), 689-692.
⁶Mao, C. B. et al., “Virus-Based Toolkit for the Directed Synthesis of Magentic and Semiconducting Nanowires,” (2004) Science, 303, 213-217.
⁷Reiss, B. D. et al., “Biological route to metal alloy ferromagnetic nanostructures” (2004) Nano Letters 4(6), 1127-1132.
⁸Huang, Y. et al., “Programmable assembly of nanoarchitectures using genetically engineered viruses” (2005) Nano Letters 5(7), 1429-1434.
⁹U.S. patent application No. 11/254,540.
¹⁰US2004/0127640
¹¹Lee, S-W. et al., “Cobalt ion mediated self-assembly of genetically engineered bacteriophage for biomimic Co-Pt hybrid material” Biomacromolecules (2006) 7(1), 14-17.
¹²Whaley, S. R. et al., “Selection of peptides with semiconductor binding specificity for directed nanocrystal assembly” (2000) Nature, 405(6787), 665-668.
¹³US2003/0148380
¹⁴US2006/0003387
¹⁵Peelle, B. R. et al., “Design criteria for engineering inorganic material-specific peptides” (2005) Langmuir 21(15), 6929-6933.
¹⁶U.S. Provisonal Patent Application 60/620, 386.

The term “target inorganic material” refers to an inorganic material that binds to a multifunctional biomolecule and can be henceforth directed to assemble to a functional structure. Such a functional structure includes, for example, a functional layer in semiconductor fabrications such as an integrated circuit layer. In one embodiment, the target inorganic material is a target integrated circuit material including but limited to: metal, metal oxide, a semiconductive material, an insulating material and a magnetic material. Advantageously, in one embodiment, the biomolecular templates' tendency to self-assemble enables an orderly construction of the target inorganic material, which makes it possible for a “bottom-up” approach in fabricating nano-sized integrated circuit components.

In one embodiment, the target inorganic material is one or more nanoparticles. The term “inorganic nanoparticle” or “nanoparticle” refers to inorganic particles of less than 100 nm in diameter. More typically, the nanoparticles are less than 50 nm in diameter, less than 25 nm in diameter or less than 10 nm in diameter. They may be crystalline, polycrystalline or amorphous.

The nanoparticles can include pre-made nanoparticles, such as colloidal gold, which can be directly conjugated to a biomolecule. Alternatively, the nanoparticles can be nucleated on a biomolecule out of a solution phase. Typically, the solution phase contains a precursor material. For example, metallic nanoparticles can be nucleated onto a peptide by reducing a precursor metal salt to the metal. In certain embodiments, reducing agents such as NaBH₄ and dimethylamine borane can be used. The metallic nanoparticles may also be nucleated without an added reducing agent when the peptide itself contains a reducing component. For example, a peptide may comprise a cysteine residue in which a free thiol group contributes to the reduction of a metal salt and subsequent nucleation of the resultant metal on the peptide.

In addition to the metallic nanoparticles described above, other examples of the inorganic nanoparticles include particles of metal oxides, semiconductive materials, magnetic materials and dielectric materials. Examples of suitable inorganic particles are summarized in Table 2.

TABLE 2

| | |
|---|---|
| Elemental conductors | Au, Ag, Pd, Mo, Cu, Fe, Co, Pt, Ru, Ni, Zn, Sn, |
| | Cr, W |
| Group IIB-VIA | ZnS, CdS, CdSe, CdTe |
| materials | |
| Group IIIA-VA | GaAs, GaN, InP, BN |
| materials | |
| Magnetic materials | Fe₂O₃, Fe₃O₄, CoPt, FePt, SmCo_{3,} Co |
| Other semi-conductive | ITO, PbS, Al₂O₃, SiNx, tantalum oxides, ZnO, |
| and conductive | CoWP, CoWB, CoP, NiWP, NiWB, NiP, carbon |
| materials | nanotubes, Si, Ge, Ce, TiO_{2,} Co₃O_{4,} HfO_{2,} |
| | indium oxide, niobium oxides, vanadium oxides, |
| | copper oxides |
| Dielectric material | SiO₂, BaTiO₃, LiNbO₃ |
| Other inorganic | CaCO₃, Ca₃(PO₃)₂ |
| material | |

In addition to the references cited previously, US 2003/0148380 entitled “Molecular Recognition of Materials” describes detailed methods of selecting and identifying biomolecules (e.g., peptides) that exhibit sequence-specific binding toward inorganic materials, including crystalline substrate and inorganic nanoparticles. The disclosure, including the sequence listing described, is incorporated herein by reference in its entirety.

(b) Biomolecules Having Material-Specific Binding Behaviors

In one embodiment, biomolecules having desired material-specific binding behaviors can be selected by combinatorial library screening. Additionally, exact binding sequences can be identified using tools and protocols developed in the field of molecular biology, such as phage display libraries.

More specifically, biological structures (e.g., a bacteriophage) that are genetically engineered can be used to express or display one or more random biomolecules, such as a peptide. For example, the biomolecule can be a random peptide of a specified length expressed as a portion of the virus' exterior coat.

The advantage of using an expression system to obtain biomolecules is that large numbers of the different biomolecules (e.g., libraries) can be provided (i.e., displayed on the phage) and screened for material-recognition, which enables rapid identification of sequences that have specific and/or selective affinity for one or more materials.

More specifically, a filamentous virus (e.g., bacteriophage) may be used to produce large numbers of one or more types of biomolecules, such as peptides. Commercially available libraries that contain random assortments of biomolecules with diversified attributes (e.g., length, innate structure, species) may also be used. For example, bacteriophage libraries (also referred to herein, as phage libraries) have been developed that include peptides of specific lengths on the minor coat protein (pIII) of the M13 coliphage. In one embodiment, a Ph.D.-12™ Phage Display Peptide Library Kit (New England BioLabs, Ipswich, Mass.) can be used. This kit contains a library with approximately 10⁹ discrete linear 12-amino acid peptide inserts fused to the pill coat protein of the M13 coliphage. In another embodiment, a Ph.D.-7™ Phage Display Peptide Library kit containing 7-amino acid peptide inserts can be used. In another embodiment, a Ph.D.-C7C™ Phage Display Peptide Library kit including disulfide constrained heptapeptides can be used. Custom designed libraries can also be used. For example, short peptide sequences can be fused onto the pVIII coat proteins of the M13 coliphage. Yeast and cell surface display libraries can also be created. See, e.g., Peelle, B. R. et al., “Probing the interface between biomolecules and inorganic materials using yeast surface display and genetic engineering,” (2005) Acta Biomateralia 1 145-154. Alternatively, such libraries can be purchased from commercial sources (e.g., the FliTrx™ random peptide library from Invitrogen, Carlsbad, Calif.).

The phage libraries can be screened against one or more materials, a process known as biopanning. Initially in the biopanning process, phages with randomized peptides are selected to have specific binding affinity for a given material and can be collected after cycles of incubation with the material and washing to remove those phages displaying peptides that are non-binding or non-specifically binding. The peptides on the phages that exhibit specific binding can be collected and used to identify the exact sequence responsible for the binding. The techniques used are those well known to one of ordinary skill in the art of molecular biology and include plating the phage or allowing various concentration of phage solution to infect a known amount of bacteria. When using the infection technique, bacteria with lacZ gene may be used and plated in the presence and absence of isopropylthio-β-D-galactoside (IPTG) and 5-bromo-4-chloro-3-hydroxyindolyl-β-D-galactose (X-gal) for visual determination of bacterial growth on “titer plates.” The phage concentration may then be determined by the following:

Concentration of phage from titer plate(pfu/μL)×(1 μl/1E⁶ L)×(1 mole/6.023×10²³ molecules),wherein,

pfu=plaque forming unit.

Several biopanning rounds are generally used to determine material-specific biomolecules and their material-specific binding sites. For each biopanning round, the phage concentration is used to determine the amount (as volume) used in the next round of biopanning against the material. A fresh piece of material is then used for the next screening, where the phage amount is at least about 10⁹ pfu. Typically, multiple rounds of biopanning are performed.

Some or all of the above steps can be automated for rapid analysis (high-throughput screening) to identify specific biomolecules that can bind or recognize a selected material with specificity and/or selectivity. These techniques are further described in detail in the following U.S. patent publications: (1) US 2003/0068900 entitled “Biological Control of Nanoparticle Nucleation, Shape, and Crystal Phase”; (2) US 2003/0073104 entitled “Nanoscale Ordering of Hybrid Materials Using Genetically Engineered Mesoscale Virus”; (3) US 2003/0113714 entitled “Biological Control of Nanoparticles”; and (4) US 2003/0148380 entitled “Molecular Recognition of Materials”; and (5) US 2004/0127640 entitled “Composition, Method and US of Bi-Functional Biomaterials”, all of which, including the sequence listings described, are incorporated herein by reference in their entireties.

Biomolecules (e.g., peptides) that successfully bind to a specific material can thus be recovered and amplified. The identity of the biomolecule can be ascertained by known techniques including isolation of the phage, sequencing its DNA and translating the DNA sequence to peptide sequence.

The peptide thus identified can also be synthesized independently of the virus, as is known in the art, with the same function and affinity as seen while displayed on the virus.

Typically, a phage-display library is based on a combinatorial library of random peptides containing between 7-12 amino acids. A peptide exhibiting specific binding to a material can be unambiguously identified by its sequence according to the process described above. Moreover, the part of a peptide sequence that in fact contributes to the binding, i.e., the binding sequence, can be determined by identifying a consensus sequence based on multiple rounds of biopanning. Additionally, screening libraries of shorter peptides against a substrate can assist with pinpointing the exact binding sequence. Furthermore, given the small size of the peptides in the phage library, computer analysis can also be used to accurately predict or confirm the identity of a binding sequence.

In another embodiment, genetically-based design can be used to produce the multifunctional biomolecules of the interest. The structural knowledge of the desired binding sequences enables a rational design of a multifunctional biomolecule, particularly with respect to multifunctional biomolecules based on peptides, proteins and polynucleotides. Well-known techniques such as site-directed mutagenesis can be used to rationally introduce modifications to one of more areas of the multifunctional biomolecules in order to produce variants. The mutation that leads to a desirable change (e.g., better specificity) in the binding characteristics can be used as a guide to work with other sequences.

Thus, through peptide (or polynucleotide) engineering, many different varieties of binding sequences can be placed at different locations on a multifunctional biomolecule. Suitable multifunctional biomolecules can therefore be designed and manufactured to combine a number of desired binding characteristics. More detailed information on genetically engineering peptide to create binding sequences are described in: e.g., Mao, C. B. et al., “Virus-Based Toolkit for the Directed Synthesis of Magnetic and Semiconducting Nanowires,” (2004) Science, 303, 213-217; Lee, S-W. et al., “Ordering of Quantum Dots Using Genetically Engineered Viruses,” (2002) Science 296, 892-895.

### 2. Labile Protecting Group

As discussed above, the biomolecular template 2 of the present invention is capable of self-assembling on the substrate 4 on account of the multifunctional biomolecule 8. In particular, the first binding site 12 of the multifunctional biomolecule has an affinity for the substrate 4 and the second binding site 14 has an affinity for a target inorganic material 20. The biomolecular template 2 further comprises the labile linkage 18 and the labile protecting group 16, which blocks the second binding site 14 of the multifunctional biomolecule 8 and is removable. The presence of the labile protecting group 16 allows for an external control of the accessibility of the second binding site 14.

In one embodiment, the biomolecular template 2 can be first deposited on the substrate 4. In response to an external stimulus, such as light, heat, enzyme or a chemical reagent that cleaves the labile linkage 18, the labile protecting group 16 is decoupled or released from the biomolecular template 2. As a result, the second binding site becomes accessible to the target inorganic material 20.

The phrase “labile protecting group” and “protecting group” are used interchangeably herein. A protecting group is labile owing to the labile linkage 18 connecting the protecting group to the biomolecular template, the labile linkage being sensitive and cleavable in response to an external stimulus. The labile linkage is otherwise stable and can withstand fabrication conditions during deposition, nucleation and plating.

Typically, the labile linkage 18 can be a chemical bond or a functionality including a chemical bond that is particularly susceptible to cleavage when subjected to light, heat, enzymatic condition or a chemical reagent. Hence, the presence of the labile linkage offers a point of manipulation of the binding activities through external means. As shown in FIG. 2B, the cleavage of the labile linkage 18 exposes the second binding site 14 for binding.

The type of the labile linkage depends on the nature of lability of the protecting group. In the case of 6-nitroveratroyloxycarbonyl derivative (NVOC), which is a protecting group cleavable by light (discussed in detail below), the labile linkage can be a carbamate group (—O—C(O)—N—) wherein the bond between the benzylic carbon and the oxygen of carbamate group is photo-cleavable. Other labile linkages derivatized from the NVOC protecting group include a carbonate group (—O—C(O)—N—) and a formate thioester (—O—C(O)—S—) group.

Alternatively, in the case of the protecting group 16 being cleavable by an enzyme, the labile linkage 18 can be a bond, e.g., a peptide bond. As will be discussed in detail below, a peptide bond formed in part by the C-terminal of an arginine is specifically recognizable and cleavable by a protease called trypsin.

A labile protecting group that can be released in response to light is also referred as being “photo-labile”. Similarly, other labile protecting groups include thermal-labile, enzymatic-labile and chemical-labile groups, which are cleavable in response to heat, enzyme and chemical agent, respectively.

(a) Photo-Labile Protecting Group

In one embodiment, the protecting group 16 of the biomolecular template 2 is a photo-labile protecting group. A photo-labile protecting group can be rapidly cleaved in response to a light irradiation.

The protection of an active site with a photo-labile protecting group is also referred to herein: as “caging”, a term typically used in experimental biology, such as cell signaling. In the context of the present invention, a binding site is an active site that can be caged by covalently attaching a photo-labile protecting group. It should be understood that, the caging process is not limited to protecting a binding site with a photo-labile protecting group. The caging process equally applies to the protection of the binding site with thermal-, enzymatic- and chemical-labile protecting groups.

The protected or “caged” binding site becomes inert until being released by flash photolysis, which cleaves off the photo-labile protecting group. This process is also referred as “uncaging”. The uncaged binding site thus becomes accessible for binding. Advantageously, the uncaging process is generally mild without the need of any harsh reagent that may potentially destabilize a biomolecule-based array. Moreover, photolysis has been widely used in semiconductor fabrication, thus the technique and apparatus (such as masks and resists) involved are well within the knowledge of one skilled in the art.

Various classes of photo-labile groups known in connection with solid-phase peptide, oligonucleotide synthesis and caged peptides are suitable for purpose of the present invention. See, e.g., Bayley, H. et al., (1997) FEBS Letters 405, 81-85; Yumoto, N. et al., (2001) Pharmacology & Therapeutics 91, 85-92; Lester, H. A. et al., (1998) Neuron 20, 619-624; and Heidecker M. et al., Biochemistry (1996) 35, 3170-3174. Most popular among these is a class of ortho-nitrobenzyl derivatives generally represented by Formula (I) below:

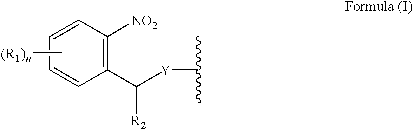

wherein:

each R₁ is the same or different and independently hydrogen, C₁₋₆ alkyl, —O—C₁₋₆ alkyl, NO₂, —CH₂COOH or —OH;

n is 0, 1, 2, 3 or 4,

R₂ is hydrogen, C₁₋₆ alkyl or —COOH; and

Y is a bond or —OC(O)—.

As used herein, C₁₋₆ alkyl refers to a saturated hydrocarbon residue having one to six carbons. The alkyl group can be branched or straight. Examples of alkyl include but are not limited to methyl, ethyl, propyl, isopropyl, butyl, t-butyl and pentyl groups.

A protecting group of Formula (I) can be coupled to a functional group of a biomolecule subunit, such as a hydroxy, a thiol or an amino group on a side chain of an amino acid. Prior to the coupling, Formula (I) can be in a reactive form. For example, a reactive form of the protecting group of Formula (I) can be 2-nitrobezylchloroformate, α-carboxy-2-nitrobezyl bromide methyl ester, 2-nitrobezyl diazoethane, 4,5-dimenthoxy-2-nitrobenzyl bromide or 2-nitrobenzyl bromide.

When Y is a bond, the protecting group can also be referred as ortho-nitro benzyl (NBz) group. U.S. Pat. No. 5,998,580 describes that all 20 natural amino acids can be modified with the NBz type of photo-labile protecting groups. This patent and the references cited therein are incorporated by reference in their entireties.

When Y is a —OC(O)— group, Formula (I) represent a photo-labile protecting group derived from ortho-nitrobenzyl alcohol, shown as Formula (II) below:

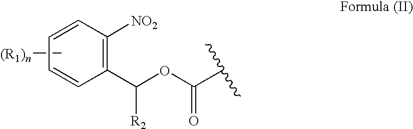

wherein, R₁, R₂, and n are as defined above.

The photo-lability of this class of protecting group is based on photo-isomerization of ortho-nitro benzyl alcohol into ortho-nitroso benzaldehyde, See, e.g., Patchornick, J. Am. Chem. Soc. (1970), 92, 6333; Amit et al., (1974) J. Org. Chem. 39, 192 and Bochet, C. G., (2002) J. Chem. Soc. Perkin Trans. 1, 125-142, which references are incorporated herein by reference in their entireties. These ortho-nitro benzyl alcohol derivatives as photo-labile protecting groups have been used in the course of optimizing the photolithographic synthesis of both peptides (see, Fodor et al. (1994) Science 251, 767-773) and oligonucleotides (see, Pease et al., Proc. Natl. Acad. Sci. USA 91, 5022-5026). See, also, US Published Application 2005/0101765; PCT patent publication Nos. WO 90/15070, WO 92/10092, and WO 94/10128; Holmes et al. (1994) in Peptides: Chemistry, Structure and Biology (Proceedings of the 13th American Peptide Symposium); Hodges et al. Eds.; ESCOM: Leiden; pp. 110-12, each of these references is incorporated herein by reference for all purposes.

The mechanism of the deprotection is shown in Scheme I below, where X represents a biomolecule moiety:

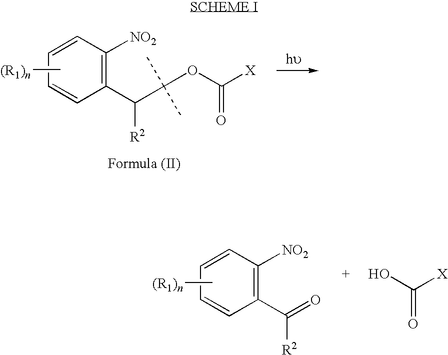

In one embodiment, the protecting group of Formula (II) is nitrobenzyloxycarbonyl group (n=0), also known as NBOC group. In another embodiment, Formula (I) represents 6-nitroveratroyloxycarbonyl group (NVOC), which incorporates two methoxy groups (R₁ is —OCH₃, n=2) in the positions meta- and para- to the nitro group. Typically, photolytic cleavage of the benzylic bond occurs at 320 nm or longer.

Other photo-labile protecting groups, such as pyrenyl system described in WO 92/10092 and t-butyl ketone system described in Kessler, M. et al., Org. Lett. (2003) 5:8, 1179-1181 can also be used. The latter has a hydroxy reactive site, which can be coupled to a carboxylic group (—COOH) of the binding site (e.g., glutamic acid or aspartic acid) and is cleavable at shorter wavelength (300 nm) than is required for ortho-nitro benzylic protecting group.

As shown in Scheme II, an example of the biomolecular template 2 of the present invention can be represented by Formula (III), wherein a NVOC is group is coupled to an amino functional group of a peptide sequence and prevents the peptide sequence from binding to a target inorganic material (R represents the rest of the biomolecule template). Upon photolysis, the protecting group is cleaved from the benzylic carbon, accompanied by spontaneous decarboxylation. As a result, a deprotected biomolecule is obtained, which is accessible for binding to the target inorganic material.

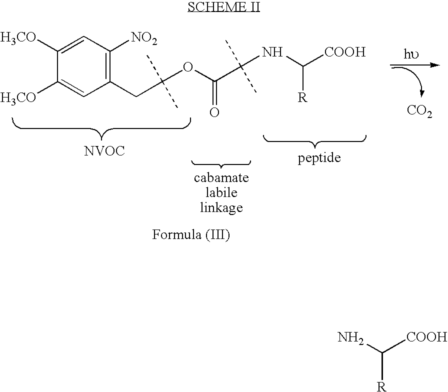

In Scheme II, a terminal amino group of a peptide is protected. However, a biomolecular template can also be protected on a side chain of an amino acid. Thus, in a similar manner, functional groups such as hydroxy group or a thiol group in the binding site of a biomolecule can be coupled to a photo-labile protecting groups described above.

In one embodiment, the protecting group 16 can be a protecting group of Formula (I) coupled to a biomolecule 8 via a functional group of the second binding site 14, thereby preventing the second binding site from binding to a target inorganic material. Examples of the suitable functional groups include an amino group of a terminal amino acid of a binding sequence, or a functional group in a side chain of an amino acid within a binding sequence, such as an amino group of a lysine, a thiol group of a cysteine or a hydroxy group of a tyrosine. These functional groups can be coupled to a reactive form of a protecting group of Formula (I) under conditions known to one skilled in the art.

Scheme III illustrates the caging and subsequent uncaging processes of a cysteine residue of a peptide suitable as the biomolecular template 2. When treated with 2-nitrobenzyl bromide, the thiol group of the cysteine is caged. The 2-nitrobenzyl moiety can be rapidly removed upon photolysis. The uncaged peptide thus is available to bind to a gold nanoparticle.

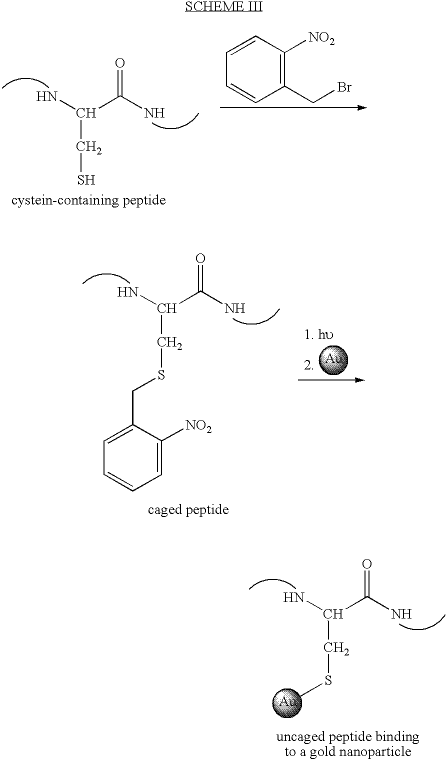

In a further embodiment, the binding intensity of a biomolecule can be manipulated by an external stimulus, such as light. Based on the same principle as illustrated in Scheme II, a biomolecule (e.g., peptide) having a caged cysteine residue can be selectively uncaged via photolysis. This process allows the freed thiol group to bind to a gold substrate. The peptide may optionally have a sequence-specific binding affinity for the substrate, and the formation of a covalent bond significantly enhances the adhesion of the peptide to the substrate.

Alternatively, a free thiol group can also be generated in situ by the reductive cleavage of a disulfide bond present in a biomolecule (e.g., peptides of the Ph.D.-C7C library from BioLab.) This process is analogous to uncaging a protected cysteine residue by photolysis illustrated in Scheme II. Here, the disulfide bond is a labile linkage connecting the cysteine residue to another cysteine residue, which can be viewed as a protecting group. The cleavage of the disulfide bond therefore leads to the uncaging of the cysteine residue. Suitable reducing agents include thiol-based reagents such as: dithiothreitol (DTT), 2-mercaptoethanol and 2-mercaptoethylamine, and phosphine-based reagent, such as Tris(carboxyethyl) phosphine (TCEP). These reagents are commercially available from Pierce Biotechnology.

Scheme III illustrates another example of manipulating the binding intensity of a biomolecule to a substrate, in which a robust adherence is achieved between the template and the substrate. Typically, the biomolecule may comprise an “adhesive group”, i.e., a functional group of the biomolecular template that forms a strong bond with the surface of the substrate. A number of functional groups can act as adhesive groups, including catechol derivatives which bind to metal surfaces such as aluminum as well as inorganic surfaces such as CaCO₃ or silicate.

Alternatively, the adhesive group can be formed in situ. In the example illustrated in Scheme III, an adhesive group is converted from a tyrosine residue present on the biomolecular template, e.g., a peptide. More specifically, the tyrosine-containing peptide can be caged by a photo-labile protecting group, such as a 2-nitrobenzyl moiety. The caged peptide can be selectively uncaged via photolysis. The freed tyrosine is then treated with tyrosine hydroxylase, an enzyme that oxidizes the tyrosine residue to L-3,4-dihydroxyphenylanaline (L-DOPA). L-DOPA is a catecholic amino acid found in the adhesive pad proteins secreted by marine mussels. L-DOPA-containing peptides therefore strongly bind to a variety of substrates, including metal and semiconductor surfaces. Accordingly, the binding intensity of a tyrosine-containing biomolecule to a substrate can be significantly enhanced by sequentially applying photolytic and enzymatic treatments.

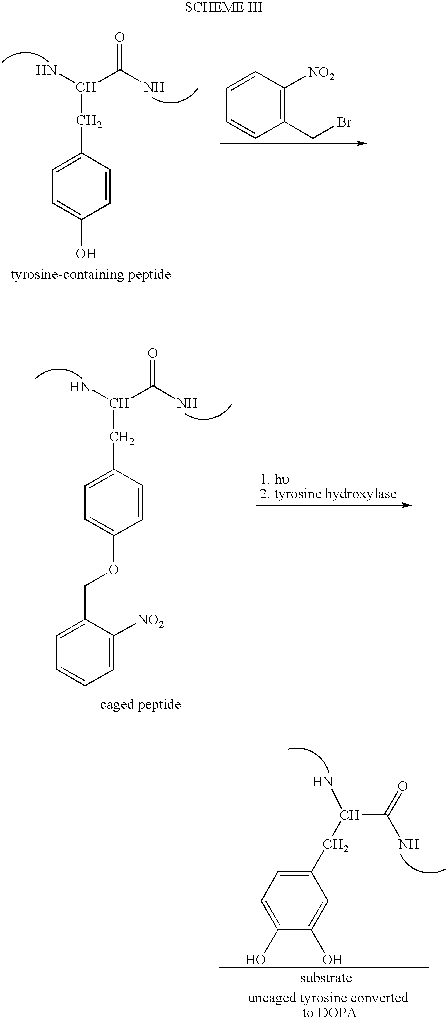

In a further embodiment, a caged reducing agent can be used in a nucleation process in which a metal salt is reduced to the metal, which subsequently nucleates on a biomolecule. The caged reducing agent affords a means to spatially manipulate the release of the reducing agent. For example, in the event that the reducing agent can be locally released near the biomolecule, the background reduction can be minimized because only the metal salt near the biomolecule will be reduced. Scheme IV illustrates a glutamic acid-containing peptide in which the glutamic acid serves the dual purposes of reducing a silver salt (e.g., CH₃CO₂Ag) in a solution to elemental silver and nucleating the silver nanoparticles. The process can be manipulated by initially caging the glutamic-acid with a photo-labile protecting group, such as t-butyl-1,2-dihydroxy-2-methylethyl ketone. The caged glutamic acid can be deprotected and the reduction capability of the glutamic acid restored. The glutamic acid henceforth converts the silver salt to silver only in the vicinity of the nucleation site.

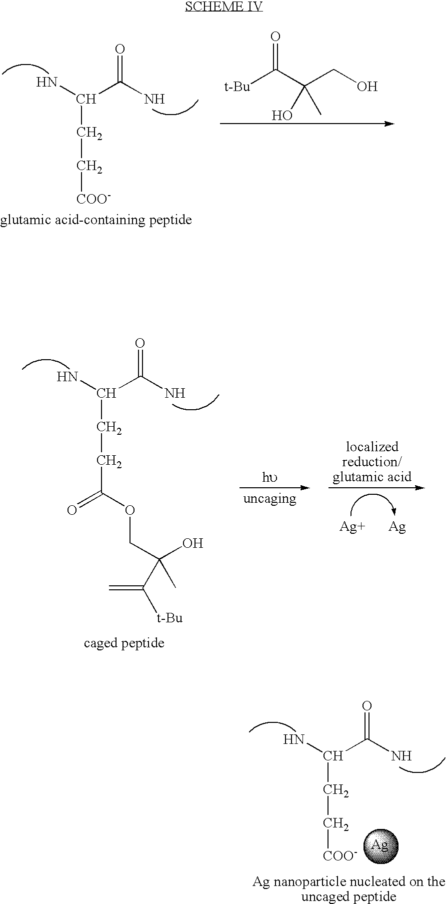

Generally speaking, the protecting group 16 can be directly coupled to the multifunctional biomolecule 8, already identified as having the desired affinity for a target inorganic material. Alternatively, the protecting group 16 can be initially coupled to a subunit (e.g., an amino acid) known to be part of the binding sequence, and be incorporated into the biomolecular template through solid-phase synthesis, during which subunits are sequentially joined together according to a selected sequence. In addition to the solid phase peptide synthesis noted above, peptides having photo-sensitive amino acid(s) can also be synthesized by biological systems. For example, a technology developed by Ambrx Inc. can provide biologically created peptides composed of un-natural amino acids, including chemically modified amino acids.

The photo-labile protecting group 16 and labile linkage 18 of the present invention are stable to a variety of reagents (e.g., piperidine, TFA, and the like); can be rapidly cleaved under mild conditions; and do not generate highly reactive byproducts. If desired, scavengers can be added to the deprotection process in order to suppress reactive byproducts, a process known to one skilled in the art.

(c) Thermal-Labile Protecting Group

In another embodiment, the protecting group 16 of the biomolecular template 2 is a thermal-labile protecting group. A thermal-labile protecting group can be cleaved in response to heat.

U.S. Pat. No. 6,699,668 and references cited therein describe a phenyl sulfoxide based protecting group, which can be coupled to a primary hydroxy group of a nucleoside. Such a protecting group is thermally cleavable and can be employed to block the second binding site of the biomolecular template described above. Moreover, Russell, H. E., et al, Thermally cleavable safety-catch linkers for solid phase chemistry. (2000) Tetrahedron Lett., 41, 5287-5290. #14621 describes a benzyl selenium oxide derivative used as a thermally cleavable protecting group in solid phase synthesis. These thermal-labile groups are suitable for protecting the second binding site via a suitable functional group, such as hydroxy, amino and thiol group. The above references are incorporated herein by reference in their entireties.

(d) Enzymatic-Labile Protecting Group:

In another embodiment, the protecting group 16 of the biomolecular template 2 is an enzymatic-labile protecting group. An enzymatic-labile protecting group can be cleaved in the presence of an enzyme. As is known, an enzyme typically recognizes a sequence-specific active site and the resulting digestion (or cleavage) is highly efficient and specific.

In one embodiment, the enzymatic-labile protecting group 16 is a portion of a biomolecule (e.g., a peptide) sequence extending from a binding sequence via a peptide bond, the presence of the protecting group 16 blocks the biomolecule 8 from binding to the target inorganic material 20. The protecting group can be cleaved in the presence of a protease that recognizes the labile linkage. Protease commonly used in analyzing protein structures as described in Kriwacki R. W. et al., Combined Use of Proteases and Mass Spectrometry in Structural Biology, (1998) J. of Biomolecular Techniques, 9:3, can be used.

For example, if arginine or lysine forms part of the labile linkage 18 (i.e., the peptide bond), trypsin, a protease specifically cleaves the C-terminal to arginine or lysine residues, can cause the protecting group 16 to decouple. Additional examples of proteases include chymotrypsin (e.g., cleaves the N-terminal to tryptophan), elastase (e.g., cleaves the N-terminal of alanine), endoprotease (e.g., cleaves the C-terminal of aspartic acid) and thermolysin (e.g., cleaves the C-terminal of leucine). Suitable proteases and identities of their specific cleavage sites are also available from commercial sources such as Pierce Biotechnology, Inc. (Rockford, Ill.).

It should be recognized by one skilled in the art, that once a binding site is identified and sequenced, a protecting group could be designed to block the binding site. The protecting group can be, for example, a short sequence of a peptide that, due to factors such as primary, secondary structure and/or localized charges, blocks or deactivates the binding site from binding to a target inorganic material.

When the multifunctional biomolecule is a polynucleotide, endonucleases can be used to cleave a specific site in the nucleotide sequence. Suitable endonucleases can be selected based on the identity of the labile linkage. Commercial vendors of endoculeases include New England BioLabs (Ipswich, Mass.).

(e) Chemical-Labile Protecting Group

In another embodiment, the protecting group 16 of the biomolecular template 2 is a chemical-labile protecting group. The chemical-labile protecting group 16 can be cleaved in the presence of a chemical reagent, including one that affects the pH of the cleavage condition. The chemical-labile protecting group therefore includes acid-labile and base-labile protecting group.

Similarly to the previously described protecting groups, the chemical-labile protecting group 16 is also coupled to the multifunctional biomolecule 8 via a functional group present in the second binding site 14 in order to block the access to the target inorganic material. Many protecting groups have been developed in connection with solid phase organic synthesis, including peptide and oligonucleotide synthesis. Protecting groups reactive toward typical functional groups present in amino acid and/or nucleotides, such as amine, hydroxy, thiol and carboxylic acid groups, have been extensively reviewed and are readily recognizable by one skilled in the art. See, Bradley, M., et al., Protecting Groups in Solid-Phase Organic Synthesis, J. Combinatorial Chemistry, (2002) 4:1, Reviews 1, which is incorporated herein by reference in its entirety.

Depending on the type of the protecting group and labile linkage, the chemical reagent utilized to decouple the protecting group also varies. Acid or base activated deprotection is commonly used for its simplicity. Other small molecule chemical reagents such as hydrazine, mercapto ethanol are also routinely employed.

Typical amine-reactive protecting groups include but are not limited to: N-fluorenylmethoxycarbonyl (Fmoc), t-butoxylcarbonyl (tBoc), Trityl, 1-(4,4-dimethyl-2,6-dioxocyclohexylidine)ethyl (Dde), phthalimide, triisopropylsulfonamide (Trs) groups. While Fmoc is base-sensitive, tBoc and Trityls are acid sensitive. Dde and phthalimide are removable by hydrazine. Removal of Trs can be readily achieved by mercapto ethanol.

Typical hydroxy-reactive protecting groups include but are not limited to: Trityl, tetrahydropyranyl, monomethoxymethyl (MOM), which are acid-sensitive. Base-sensitive hydroxy protecting groups include but are not limited to acytyl, benzoyl (Bz), 2,2,2-tricholoethoxycarbonyl (Troc) and Fmoc.

Esterification is typically used to protect a carboxylic acid moiety of a binding site by forming methyl or ethyl esters therein. De-esterification under acidic or alkaline conditions is known to one skilled in the art.

Typical thiol-reactive protecting groups include but are not limited to: trityl (acid sensitive), acetyl (base sensitive) and ethyl (dithiolthreitol sensitive).

The acid-labile protecting groups described above can be selectively deprotected by an acid generated in situ upon exposure to photo-irradiation. The term “photoacid generator” (PAG) refers to a photosensitive material that forms an acid moiety upon exposure to a light source. Any materials that can generate an acid moiety upon irradiation are suitable for the present invention. In particular, suitable PAGs can be those typically used in combination with chemically amplified resists in photolithographic applications, See, e.g., U.S. Pat. Nos. 5,212,043 and 6,132,926, WO 97/33198, WO 96/37526, EP 0 794 458 and EP 0 789 278. Examples of the photoacid generators include sulfide and onium type compounds. In one particular embodiment of the present invention, the photoacid generator is diphenyl iodide hexafluorophosphate, diphenyl iodide hexafluoroarsenate, diphenyl iodide hexafluoroantimonate, diphenyl p-methoxyphenyl triflate, diphenyl p-toluenyl triflate, diphenyl p-isobutylphenyl triflate, diphenyl p-tert-butylphenyl triflate, triphenylsulfonium hexafluororphosphate, triphenylsulfonium hexafluoroarsenate, triphenylsulfonium hexafluoroantimonate, triphenylsulfonium triflate, dibutylnaphthylsulfonium triflate or mixtures thereof.

The base-labile protecting groups of the present invention can be likewise deprotected by exposure to a photo-irradiation in the presence of a photobase generator. The term “photobase generator” (PBG) refers to a photosensitive material that forms a base moiety upon exposure to a light source. An example of PBG is N-2-nitro-4,5-dimethoxybenzyloxycarbonyl-cyclohexylamine. Upon exposure to ultraviolet irradiation (e.g., at 365 nm), N-2-nitro-4,5-dimethoxybenzyloxycarbonylcyclohexylamine produces cyclohexylamine, which is a mild base. More examples of PBG are described in, for example, U.S. Pat. No. 6,045,977.

Additional examples of PAGs and PBGs can be found in the following review article by M. Shirai, et al., “Photoacid and photobase generation in photoresists”, (1999) Photochemistry & Photobiology, 5, 169-185. Many commercially available PAGs and PBGs are suitable for a variety of near- and deep-UV irradiation sources, including mercury g-line (436 nm), h-line (405 nm), 1-line (365 nm), KrF laser (248 nm), ArF laser (193 nm), etc.

As will be described in more detail below, PAGs (or PBGs) causes the acid-labile protecting group (or base-labile protecting group) to decouple from the biomolecular template 2 only when the biomolecular template 2 is exposed to a light source. This mechanism of selective deprotection allows for a patterned formation of a target inorganic material by exposing the biomolecular templates to light irradiation according to a desired pattern. One skilled in the art readily recognizes that the wavelength of the light irradiation dictates in part the resolution of the pattern formed. It is therefore within the knowledge of one skilled in the art to select a PAG (or PBG) associated with an irradiation source of a desired wavelength.

### 3. Method of Using the Biomolecular Templates Having Labile Linkages

In one embodiment, the biomolecular templates described herein can be used to plate a layer of a target inorganic material on a substrate based on the “bottom-up” approach.

FIG. 2B illustrates schematically a method of conjugating a target inorganic material using the biomolecular template 2 described above. The method comprises initially depositing the biomolecular template 2 on the substrate 4, the biomolecular template 2 having the multifunctional biomolecule 8 including the first binding site 12 coupled to the substrate 4 and the second binding site 14 having an affinity for the target inorganic material 20. The multifunctional biomolecule 8 is further coupled to the protecting group 16 via the labile linkage 18 such that the second binding site 14 is prevented from binding to the target inorganic material 20. Next the biomolecular template 2 are subjected to an external stimulus, such as light, to cause the labile linkage 18 to degrade and the protecting group 16 removed. Thereafter, the substrate is contacted with a fluid containing the target inorganic material 20, wherein the target inorganic material 20 is conjugated to the second binding site 14. A biomolecular conjugate 22 is obtained after the steps of deprotection and conjugation.

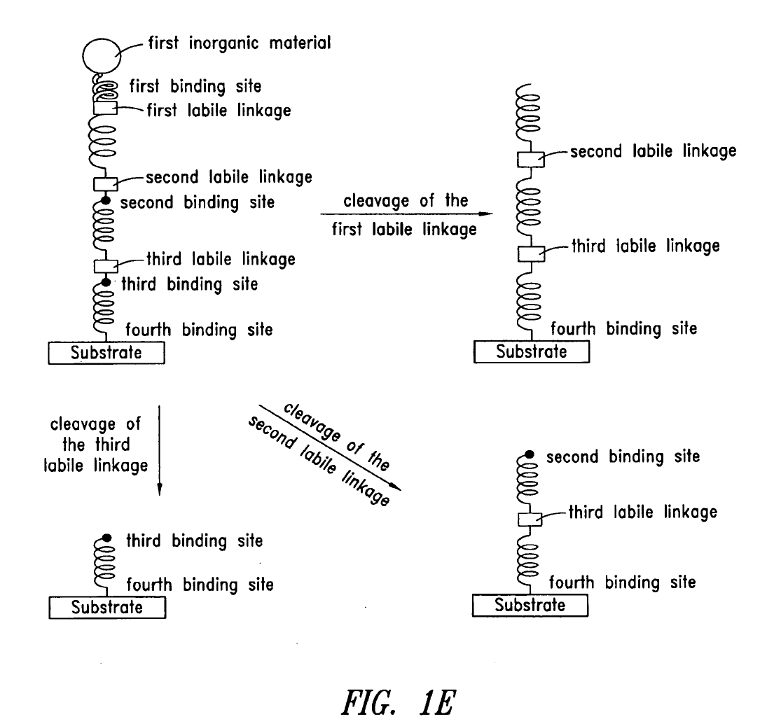

FIGS. 3A-3C illustrate an application of the biomolecular template 2 according to a further embodiment of the present invention, wherein an inorganic material layer can be formed and patterned on a substrate through a layer of biomolecular templates. According to this embodiment, the method comprises: depositing a plurality of biomolecular templates on the substrate to form a template layer, each said biomolecular template having a multifunctional biomolecule including a first binding site coupled to the substrate and a second binding site having an affinity for the target inorganic material, and a protecting group coupled to the multifunctional biomolecule via a labile linkage such that the second binding site is prevented from binding to the target inorganic material; subjecting, according to a selected pattern, a region of the template layer to an external stimulus; deprotecting the second binding sites of the biomolecular template in the region subjected to the external stimulus by degrading the labile linkages thereof; and contacting the substrate with a fluid containing the target inorganic material, wherein the target inorganic material is conjugated to the second binding sites in the region.

The patterned formation of a target inorganic material in accordance to this embodiment has a number of advantages, including but are not limited to those listed below. First, the patterning is accomplished at a better resolution, especially, when the external stimulus is light. Secondly, the method described herein, although akin to photolithography, no photo-resist is required. Thirdly, the “bottom-up” approach based on biomolecule-directed assembly is economical by eliminating the etching process typically associated with the “top-down” technique, thereby incurs no waste of the target inorganic material. Finally but not the least, by incorporating a labile protecting group in the biomolecular template, the formation of an organic-inorganic interface can be modulated or controlled through an external means, such as light, heat, enzyme and chemical reagent.

In one embodiment, a mask is used to create a desired pattern according to which an inorganic material is conjugated on a layer of biomolecular templates. More specifically, as shown in FIG. 3A, a plurality of biomolecular templates 2 can be deposited on a substrate 4 to form a template layer 24, the first binding site 12 of the biomolecular template 2 being coupled to the substrate 4. As discussed above, each second binding site 14 is blocked by the respective labile protecting group 16, in this case, a photo-labile protecting group. The external stimulus is from a light source 28 that provides a light irradiation 32. A photo-mask 30 is positioned above and covers a region 34 of the template layer 24 according to a selected pattern that leaves exposed regions 36 and 36′ of the template layer 24. The template layer 24 is then exposed to the light irradiation 32, which cleaves the labile linkage 18 in the exposed regions 36 and 36′ but does not affect the biomolecular template 2 in the region 34 protected by the mask 30. Cleaving those labile linkages 18 removes the photo-labile protecting groups 16 in the regions 36, 36′ and exposes the underlying second binding sites.

As shown in FIG. 3B, after the removal of the mask 34, the biomolecular templates 2 have been converted to multifunctional biomolecules 8 in the regions 36 and 36′, but remain intact in the formerly masked region 34. As further illustrated in FIG. 3C, contacting the substrate 4 with a fluid containing the target inorganic material 20, causes the accessible second binding sites in the regions 36 and 36′ to be conjugated to the inorganic material to form biomolecular conjugates 22. Hence, a patterned target inorganic material layer 26 is formed in the regions 36 and 36′, as defined by the selected pattern of the mask 30.

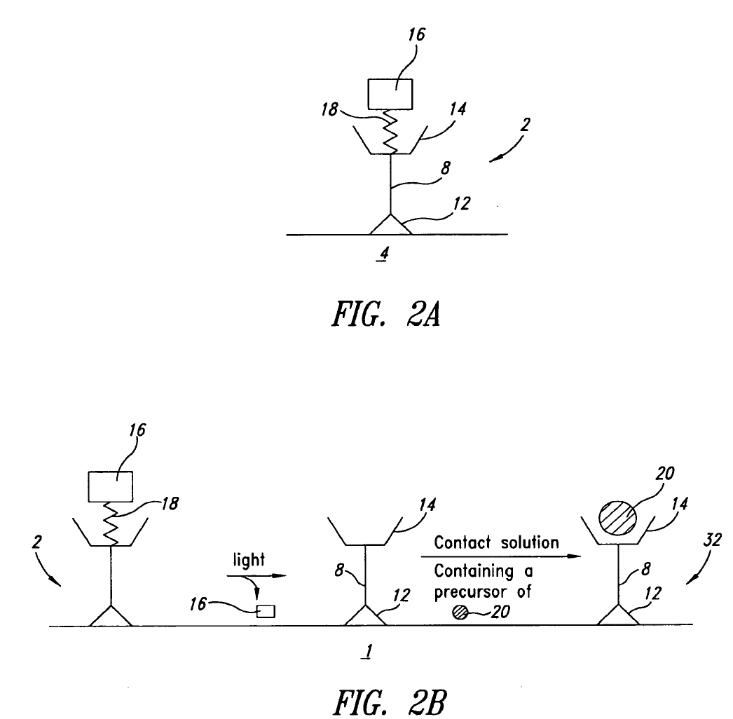

In another embodiment, an insulating thermal-mask can be used to create a desired pattern using heat as the external stimulus. More specifically, a plurality of biomolecular templates 40 are deposited on the substrate 4 to form a template layer 42, as shown in FIG. 4A. The biomolecular template 40 has the same structure as the biomolecular template 2 discussed above, except that the biomolecular template 40 has a thermal-labile protecting group 43 rather than the photo-labile protecting group 16. The external stimulus can be a flood IR source that generates heat radiation 44. An insulating thermal mask 45 is positioned above the template layer 42 and covers a region 54 while exposing regions 56 and 56′ to the heat radiation 44.

Alternatively, a light-absorbing, heat transfer layer disposed between a photo-mask and the template layer can be used to convert a light irradiated region into a heated region. As shown in FIG. 4B, a heat transfer layer 46 is positioned between the template layer 42 and a photo-mask 48, the template layer 42 being formed as described above. Analogous to the thermal-mask 45, the photo-mask 48 covers a region 54 while exposing regions 56 and 56′ to a light irradiation 32. Upon exposure to the light irradiation 32, the heat-transfer layer 46 converts the photon energy into thermal energy in regions 56 and 56′ only.

In response to the localized heat in regions 56 and 56′, the biomolecular templates 40 in these regions are deprotected due to the cleavage of the thermal-labile groups 43 (as shown in FIG. 4C).

Following the removal of the thermal-mask 45 or the photo-mask 48 and the heat-transfer layer 46, the substrate 4 is dipped in or otherwise contacts a fluid containing the inorganic material 20. The second binding site 14, now accessible in regions 56 and 56′, are coupled to the inorganic material 20 to form the biomolecular conjugates 22 (as shown in FIG. 4D). Hence, the biomolecular templates 40 direct the formation of a target inorganic material layer 60 in the regions 56 and 56′, as defined by the selected pattern of the thermal-mask 45 or photo-mask 48.

In a similar manner, biomolecular templates having enzymatic-labile protecting groups can be selectively deprotected according to a desired pattern using a mask. As a result of the mask, only selected regions of biomolecular templates come to contact or are exposed to an external stimulus, as illustrated in FIGS. 3A-3C and FIG. 4A-4D.

Likewise, biomolecular templates having chemical-labile protecting groups can be selectively deprotected according to a desired pattern using a mask. Particularly with respect to acid-labile or base-labile protecting groups, a photoacid generator (or photobase generator) can be used in conjunction with a photo-mask.

As an alternative to using the masks to create a desired pattern, a mask-less operation, such as soft lithography, can be used to directly transfer or “print” the biomolecular templates on a substrate according to the desired pattern. See, e.g., Xia, Y. et. al, (1998) Soft Lithography. Angew. Chem. Int. Ed. Engl. 37, 551-575; and Xia, Y. et. al, (1998) Soft Lithography Annu. Rev. Mater. Sci. 28, 153-184. Soft lithography refers to a set of technologies for micro- or nano-fabrication, including microcontact printing, replica molding, microtransfer molding, micromolding in capillaries and solvent-assisted micromolding. Soft lithography is based on printing and molding using elastomeric stamps with the patterns of interest in bas-relief. The technique is particularly suited for transferring biological materials. In brief, a stamp having the desired pattern can be created. The stamp is typically made of a resin material, including fluorosilicone. The stamp is then “inked” by incubating it, pattern-up, into a solution of the biomolecular templates. The biomolecular templates will adsorb to the stamp, typically in a single layer. The inked stamp is then pressed onto a substrate and removed, leaving a patterned layer of the biomolecular templates where the pattern on the stamp contacted the substrate.

In one embodiment, the target inorganic material is a nanoparticle. Suitable nanoparticles include metals, metal oxides, metal alloys, dielectric materials and magnetic materials, as those described above. Furthermore, nanoparticle nucleation on a biomolecule-based template has been described in detail in the following U.S. patent publications: (1) US 2003/0068900 entitled “Biological Control of Nanoparticle Nucleation, Shape, and Crystal Phase”; (2) US 2003/6073104 entitled “Nanoscale Ordering of Hybrid Materials Using Genetically Engineered Mesoscale Virus”; (3) US 2003/0113714 entitled “Biological Control of Nanoparticles”; and (4) US 2003/0148380 entitled “Molecular Recognition of Materials”; (5) US 2004/0127640 entitled “Composition, Method and US of Bi-Functional Biomaterials” and (6) US 2005/0064508 “Peptide Mediated Synthesis of Metallic and Magnetic Materials”, which references are incorporated herein by reference.

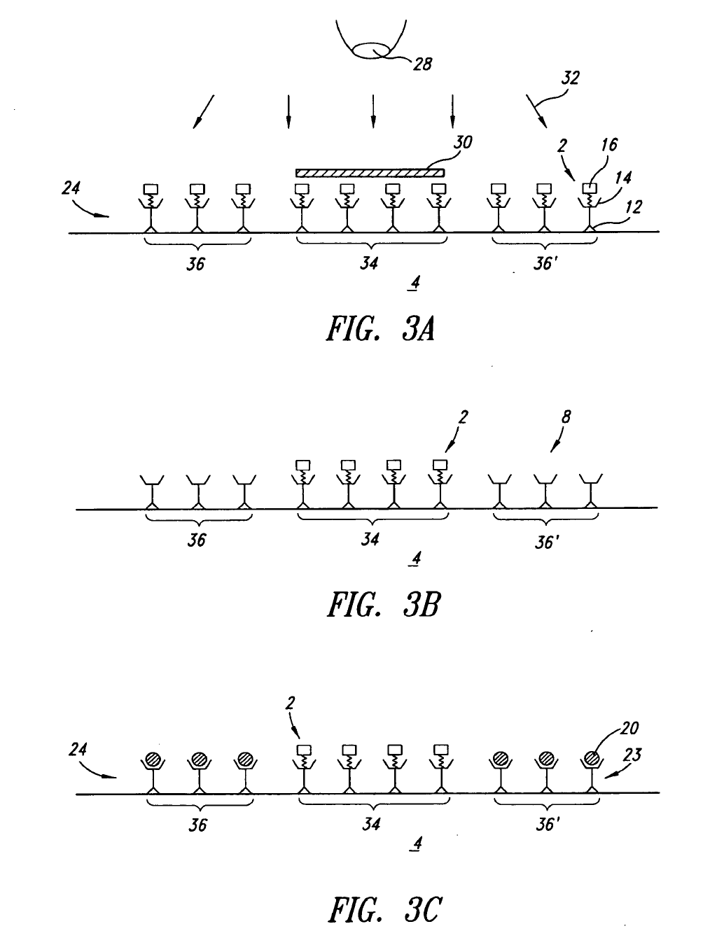

As noted above, a nanoparticle bound to a multifunctional biomolecule can further nucleate the growth of a target inorganic material. In one embodiment, as schematically illustrated in FIG. 5, using the patterned target inorganic material layer 60 of FIG. 4D as a seed layer 62, a second target inorganic material layer 66 can be deposited on the seed layer 62 according to the same pattern thereof. More specifically, the seed layer 62 comprising first nanoparticles 20 is dipped in a solution containing a precursor of the target inorganic material 64. In one embodiment, the second target inorganic material is metal, the precursor can be a salt of the metal. The first nanoparticle 20 catalyzes the reduction of the precursor (e.g., the metal salt) and enables the growth and plating of the second inorganic material 64.

The first nanoparticle 20 and the second target inorganic material 64 can be the same or different. For example, gold (Au) nanoparticles are capable of catalyzing the reduction of CuSO₄ (a precursor of copper) to copper (Cu). Au nanoparticles can be initially coupled to the second binding site 14 of the deprotected biomolecular templates 8, according to a process illustrated in FIGS. 3A-3C and FIGS. 4A-4D. As further illustrated in FIG. 5, the substrate 4 having the Au seed layer 62 can be dipped in a CuSO₄ solution, thereafter, the Au layer 62 acts as a seed layer to nucleate a Cu layer 66.

Other examples of the first nanoparticles 20 that can be used as the seed material include Ni, Cu, Pd, Co, Pt, Ru, Ag, Cr, Mo, W, Co alloys or Ni alloys. The second inorganic material 64 that can be subsequently plated include metals, metal alloys and metal oxides, for instance, Cu, Au, Ag, Ni, Pd, Co, Pt, Ru, Ag, Cr, W, Mo, Co alloys (e.g., CoPt, CoWP), Ni alloys (e.g., NiP, NiWP), Fe alloys (e.g., FePt) or TiO₂, CO₃O₄, Cu₂O, HfO₂, ZnO, vanadium oxides, indium oxide, aluminum oxide, indium tin oxide, nickel oxide, copper oxide, tin oxide, tantalum oxide, niobium oxide, vanadium oxide or zirconium oxide. More details of using seed layers to direct functional layer formations are described in co-pending U.S. provisional application No. 60/680,491, entitled “Biologically Directed Seed Layers and Thin Films”, filed May 13, 2005, in the name of Cambrios Technologies, which reference is incorporated herein in its entirety.

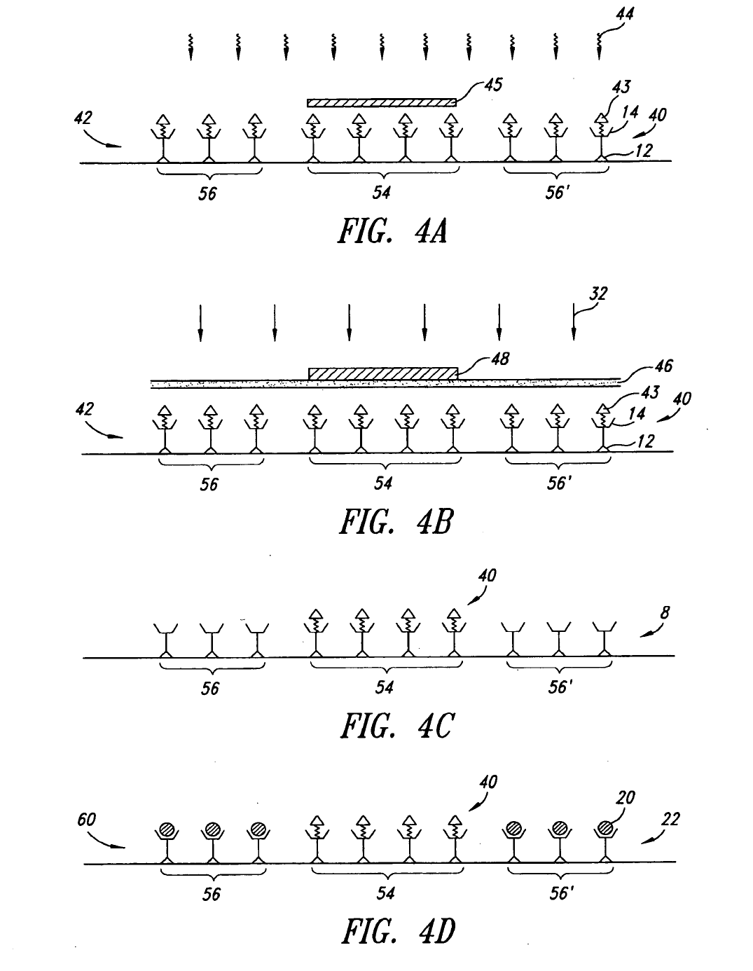

As an alternative to using a mask, a selected pattern can be directly “written” on a layer of biomolecular templates. More specifically, a laser direct-write or e-beam lithograph can introduce a pattern in a template layer by exposing only certain regions of the template layer to a laser beam. Rather than utilize a mask to form a pattern, the pattern is typically formed using a raster scan process, during which the laser is moved over a surface of the template layer and only turned on over designated regions according to a desired pattern. As illustrated in FIG. 6A, the biomolecular templates 2 form the template layer 24 on the substrate 4. As described above, each biomolecular templates 2 comprises the multifunctional biomolecule 8 having the first binding site 12 and the second binding site, and a photo-labile protecting group 16 coupled to the multifunctional biomolecule 8 via a labile linkage 18. A laser source 70 irradiates only selected regions 76 and 76′. The biomolecular templates 2 in regions 76 and 76′ are therefore exposed to the light and become accessible for binding to the target inorganic material 20 to form a patterned target inorganic material layer 78 (shown in FIG. 6B).

Because the binding event occurs on a molecular level following the cleavage of the labile linkage of each template, the patterning process is capable of creating nanometer-scaled feature sizes. Accordingly, high-resolution patterning can be achieved.

As a further alternative to the mask-less approach of patterning described above, holographic exposure or interference lithography can be used. Interference lithography is a standard technique for making gratings and point arrays, which typically uses flood exposures without the need for masks. Periodic patterns with very high resolution and regularity can be achieved. see, e.g., “Optical technique for producing 0.1-μ periodic surface structures” by C. V. Shank and R. V. Schmidt, Appl. Phys. Lett., Vol. 23, No. 3, 1 Aug. 1973, pp. 154-155.

In the above embodiments, biomolecular templates are initially deposited on the substrate, as defined herein. Direct deposition can be typically achieved by contacting the biomolecular templates in a solution phase with the substrate. Simply put, the substrate can be dipped into a solution of the biomolecular templates.

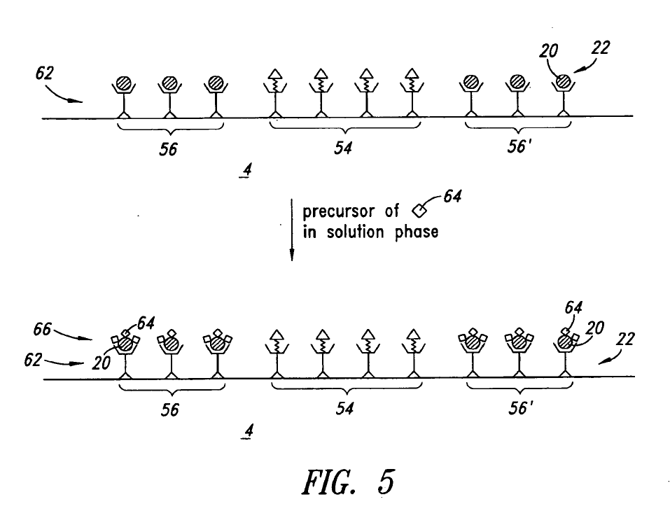

Alternatively, the biomolecular templates can be directly printed on the substrate according to the methods described in U.S. patent Ser. No. 11/280,986, entitled “Printable Electronics”, filed on Nov. 16, 2005, in the name of Cambrios Technologies, the assignee of the present invention, which application is incorporated herein by reference in its entirety. As schematically illustrated in FIG. 7A, an ink layer 80 can be printed on the substrate 4. The ink layer is selected such that it has an affinity for the first binding site 12 of the biomolecular template 2. The biomolecular templates 2 therefore self-assemble on the ink layer 80. More typically, a seed material 82 is first adhered to or incorporated in the ink layer 80, as illustrated in FIG. 7B. The first binding site 12 has affinity for the seed material 82, which directs the assembly of the biomolecular templates 2 on the substrate.

As discussed above, biomolecular templates having labile protecting groups can direct the formation of a target inorganic material on a substrate according to a desired pattern. This aspect of the invention finds numerous applications ranging from forming patterned layers in electrical circuit fabrications to creating plasma displays. For example, when the target inorganic material includes metal nanoparticles, biomolecular templates of the present invention can be metalized according to a desired pattern to form an interconnect layer or a bus line layer.

### 4. Biomolecular Conjugates Having a Labile Linkage and Method of Using Thereof

In one embodiment, the present invention provides a biomolecular conjugate comprising a multifunctional biomolecule (e.g., a bifunctional biomolecule) having a first binding site having an affinity for a substrate, a labile linkage and a second binding site, and a target inorganic material coupled to the second binding site.

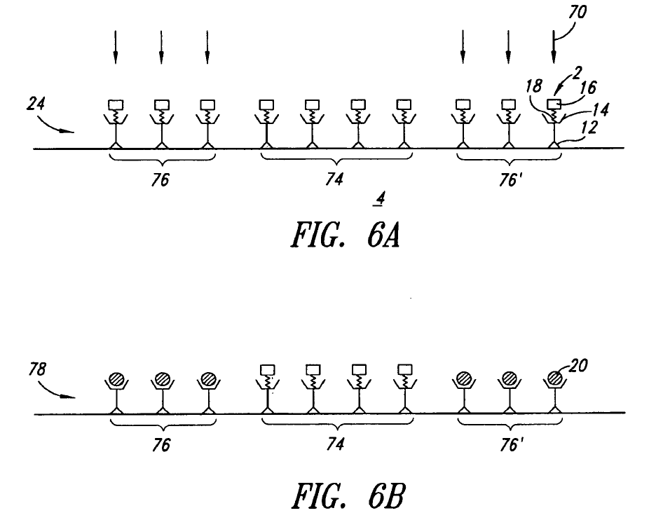

As shown in FIG. 8, a biomolecular conjugate 88 comprises a multifunctional biomolecule 90 having a first binding site 92, a second binding site 94 and a labile linkage 96. The first binding site 92 has specific and/or selective affinity for the substrate 4. The second binding site 94 is further conjugated to a target inorganic material 100. The phrases “first binding site” and “second binding site” are as defined previously.

The labile linkage 96 offers a controlled means to mediate the organic-inorganic interface in the biomolecular conjugate. More specifically, the labile linkage can be triggered by an external stimulus with the result of disrupting the binding behavior of the second binding site 94, which in turn causes the removal of the target inorganic material 100.

Typically, a labile linkage is a part of a biomolecule conjugate and is sensitive or reactive to an external stimulus. In one embodiment, the labile linkage degrades and causes detachment of a binding site, either in whole or in part. For example, a labile linkage can be an integral part of the backbone of a multifunctional biomolecule, such as a peptide bond of a peptide sequence. External stimuli, such as an enzyme, can target one or more amide bonds of the biomolecular conjugate to cleave the binding site. Suitable proteases that recognize specific peptide bonds are described in Kriwacki (supra). In addition, a metal complex with or without light irradiation has been reported as a means to cleave specific peptide bonds. Pedersen, P. L. et al. “Novel Insights of the Chemical Mechanisms of ATP Synthase” (1997) J. Biol. Chem. 272:30 p. 18875. See, also, Grant K. B. et al. “Selective Hydrolysis of Peptides Promoted by Metal Ions: a Positional Scanning Approach.” (2002) Chemical Communications 14, 1444-1445. Furthermore, it is well known to one skilled in the art that heat induces denaturing of peptides, albeit in a less selective manner than typical enzymatic cleavage of a peptide bond.

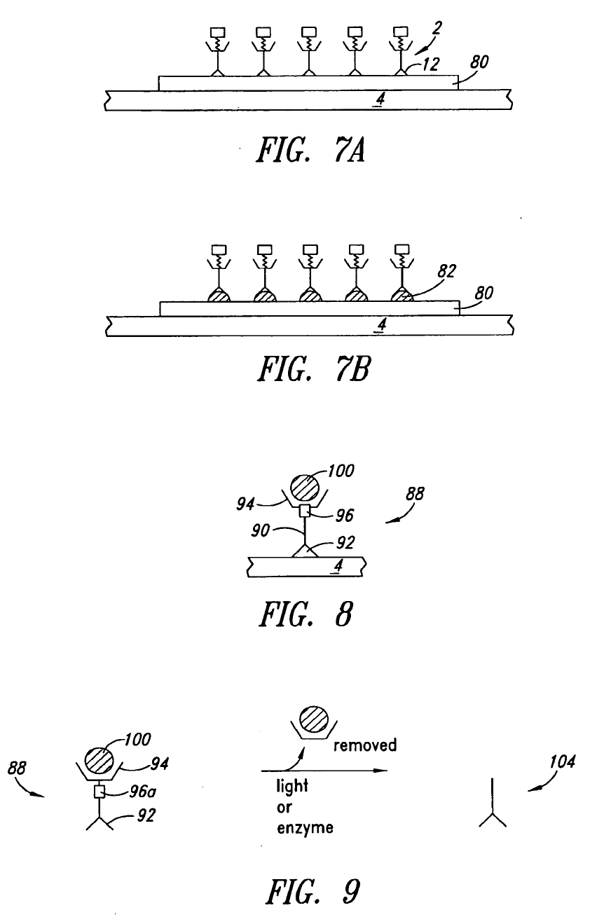

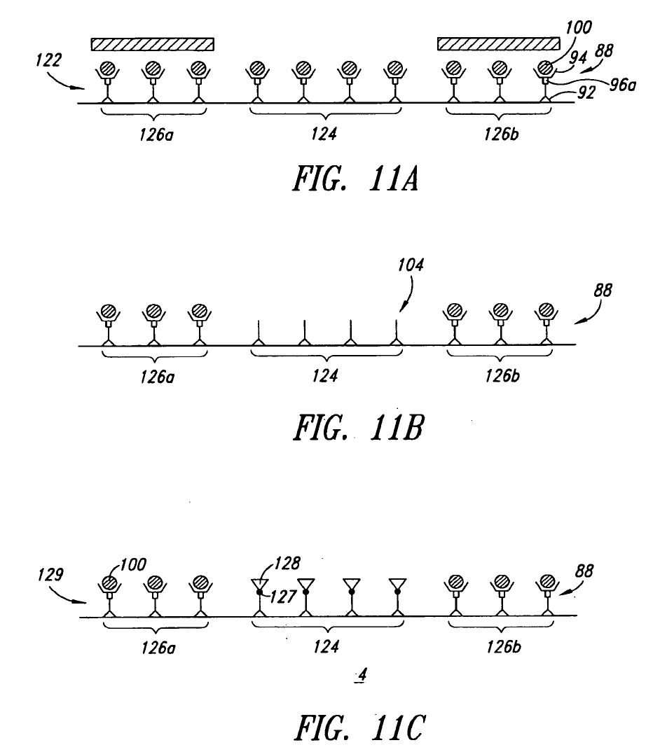

FIG. 9 illustrates schematically the removal of the target inorganic material through cleaving the labile linkage that removes the second binding site. A labile linkage 96a, such as a photosensitive linkage, is present in the biomolecular conjugate 90. The labile linkage 96a is not necessarily involved in the active binding to the target inorganic material 100. When exposed to an external stimulus, such as light, the photosensitive labile linkage 66a degrades and causes the second binding site 94 and the target inorganic material 100 to be cleaved. Likewise, if the labile linkage 66a is enzymatic-sensitive, an appropriate enzyme will cleave the labile linkage 66a. The remainder biomolecule 104 represents an “etched” form of the biomolecular conjugate 88 from which the target inorganic material 100 has been removed. Advantageously, as further illustrated in FIGS. 11A-11B, described below, this etching process can be applied to patterning a target inorganic material layer by manipulating the external stimulus according to a selected pattern.

In other embodiments, the labile linkage may be part of a binding sequence and is susceptible to being modified. As a result, the binding activity of the binding sequence is disrupted and the inorganic material previously conjugated can be removed. For instance, a labile linkage can be a critical functional group that contributes to binding to a target inorganic material. Exposure to an external stimulus may modify the labile linkage in such a way that the binding is no longer possible. For example, it has been found that amino acids having positively charged side chains (e.g., arginine, histidine) exhibit an affinity for ZnS. Under circumstances in which a chemical agent deprotonates the positively charged side chains, the binding will be disrupted. In one embodiment, the labile linkage is a histidine, in particular, the positively charged imidazole ring of histidine. Exposure to a chemical reagent deprotonates histidine therefore leads to the disruption of the binding activity of the histidine-containing binding sequence.

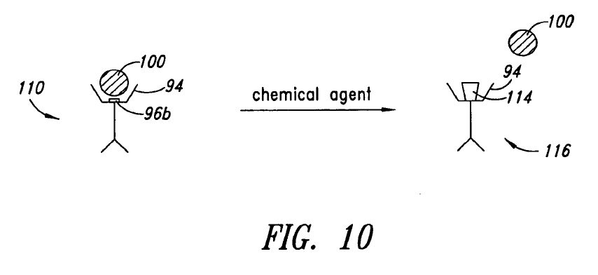

FIG. 10 shows schematically the removal of an inorganic material as a result of a disruption of a binding site. A biomolecular conjugate 110 comprises a labile linkage 66b as part of the binding sequence 94 that is conjugated to the target inorganic material 100. The labile linkage 66b is actively involved in binding to the inorganic material 100. Upon exposure to a chemical agent, labile linkage 66b is transformed to a modified form 114, which renders the second binding site 94 unable to bind to the inorganic material 100. The remainder 116 represents an “etched” form of the initial biomolecular conjugate 110 from which the inorganic material 100 has been removed.

In a further embodiment, the present invention provides a method of patterning a target inorganic material layer composed of a plurality of nanoparticles, comprising: depositing a layer of biomolecular conjugates on a substrate, each biomolecular conjugate including a multifunctional biomolecule having a first binding site coupled to the substrate, a labile linkage and a second binding site, and a nanoparticle coupled to the second binding site of the multifunctional biomolecule; subjecting, according to a selected pattern, a region of the biomolecular conjugates to an external stimulus; and detaching the nanoparticles from the biomolecular conjugates in the region subjected to the external stimulus.

Accordingly, an inorganic material layer comprising inorganic nanoparticles coupled to biomolecular conjugates can be patterned by a selective removal or “etching” of the inorganic nanoparticles according to a selected pattern. Similar to the patterned formation of an inorganic material layer using biomolecular templates according to a selected pattern through manipulation of an external stimulus, as described above, “etching” of an inorganic material from biomolecular conjugates can be achieved by subjecting the biomolecular conjugates to an external stimulus. Accordingly, similar methods of identifying binding sequences, localized light irradiation, heating, localized enzymatic and chemical treatments can be used. Likewise, biomolecule deposition methods as described above can be used to deposit the biomolecular conjugates on a substrate.

In one embodiment, a mask can be used to direct the external stimulus according to a selected pattern. As illustrated in FIG. 11A, the mask 120 is positioned above a target inorganic material layer 122 comprising a plurality of inorganic nanoparticles 100 present in the biomolecular conjugates 88, the structure of which is as described above. A region 124 of the inorganic material layer 120 is treated with an enzyme, e.g., trypsin, while regions 126a and 126b are masked and not exposed to the enzyme. As further illustrated in FIG. 11B, the labile linkage 96a is cleaved by the enzyme in the region 124 only, which results in the “etching” of the target inorganic material layer 122 in region 124.

In a further embodiment, the cleavage of the labile linkage 96a of the biomolecular conjugate 88 reveals a third binding site 127 (shown in FIG. 11C). The third binding site can be further conjugated to a second target inorganic material 128. According to this embodiment, a multi-functional target material layer 129 is obtained. The layer 129 comprises two types of the target inorganic materials 100 and 128 in different regions.

The above methods provide an alternative approach to the traditional lithographic method of fabricating electrical circuit components. The method described herein relies on a biomolecule-directed assembly of inorganic nanoparticles to form a target inorganic material layer. The etching step is carried out by the controlled removal of the inorganic nanoparticles according to a selected pattern. The inorganic nanoparticles are therefore “etched” as a result of a disruption in the binding behavior of the binding site in response to an external stimulus, such as light, heat, enzymes and chemical reagents.

In a further embodiment, a method is provided herein to form a target inorganic material layer according to a desired pattern, the method comprising: depositing a layer of multifunctional biomolecules on a substrate, each multifunctional biomolecule including a first binding site coupled to the substrate, a labile linkage and a second binding site having an affinity for the target inorganic material; subjecting, according to a selected pattern, a region of the multifunctional biomolecules to an external stimulus; removing the second binding sites from the multifunctional biomolecules in said region by cleaving the labile linkages thereof; and contacting the substrate to the target inorganic material whereby the target inorganic material binds the second binding sites of the multifunctional biomolecules in a region not exposed to the external stimulus.

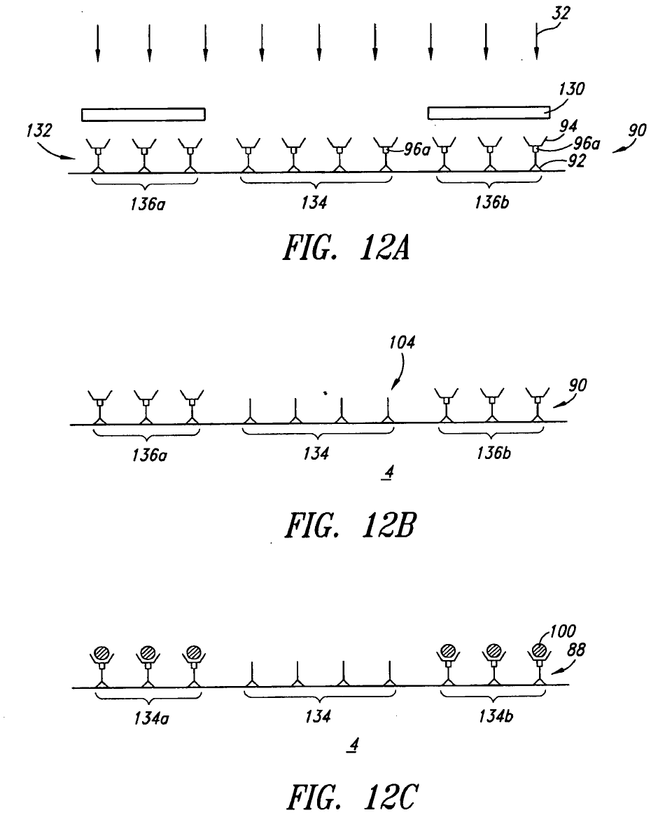

As shown in FIG. 12A, a layer 132 of the multifunctional biomolecules 90 is deposited on the substrate 4. Each multifunctional biomolecule 90 comprises a first binding site 92, a labile linkage 96a and a second binding site 94 (see, also, FIG. 8). A mask 130 is positioned above the biomolecule layer 132, defining a region 134 to be exposed to an external stimulus, in this case, light irradiation 32. Regions 96a and 96b are masked.

As shown in FIG. 12B, the multifunctional biomolecules 90 in region 134 are cleaved at the labile linkages 96a thereof with the result of removing the second binding sites 94.

FIG. 12C further illustrates that, after the removal of the mask 130, the substrate 4 can be contacted with the target inorganic material 100. Only those multifunctional biomolecules having intact binding sites in the regions 96a and 96b will bind to the inorganic material 100. Thus, a pattern of the inorganic material 100 is created.

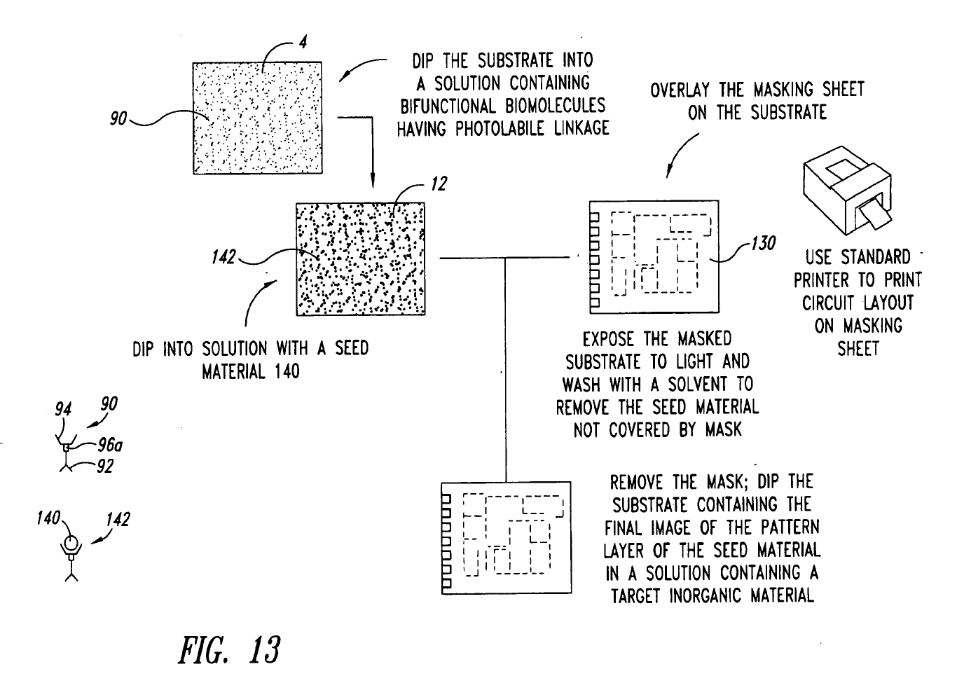

FIG. 13 illustrates a further embodiment according to principles of the present invention. In the embodiment of FIG. 13, a patterned layer of a seed material is first formed on the substrate 4 on which it is desired to form an electrical circuit. For example, the electrical circuit comprises a target inorganic material, such as a conductive material, which will nucleate around the seed material. More specifically, the substrate 4 is first dipped into a solution containing multifunctional biomolecules 90. The structures of the multifunctional biomolecules 90 are as described above. The multifunctional biomolecules are bound to the substrate 4 in a blanket layer across the substrate due to their affinity thereto. Subsequently, the substrate 4 is dipped into a solution containing a seed material 140. The seed material is conjugated to the multifunctional biomolecule to form a biomolecular conjugate 142. Next, a masking sheet 130 of a desired pattern, e.g., an electrical circuit layout, is overlaid on the substrate 4. The masking sheet 130 can be printed using a standard printer, such as a laser printer, inkjet printer, or any other acceptable printer. Thereafter, the masked substrate is exposed to a light irradiation. The seed material 142 uncovered by the masking sheet 130 is cleaved and can be removed by washing the substrate with an appropriate solvent. The seed material covered by the masking sheet 130 remains and the substrate now contains the final image of the patterned layer of the biomolecular conjugate 142 (multifunctional biomolecules 90 coupled to the seed material 140). Finally, the substrate is dipped into a solution containing a precursor of the target inorganic material. A patterned layer of the target inorganic material can be formed by plating the target inorganic material on the seed material, as previously described. The target inorganic material can be, for example, metallic nanoparticles that are capable of forming bus lines according to a desired pattern.

In a further embodiment, two or more of the above methods of patterning can be combined to create complex patterns through serial manipulations of external stimuli.

It is noted that in all of the above embodiments, the biomolecules can be removed from the material they are bound to by thermal annealing or sintering. U.S. patent application Ser. No. 10/976,179 and Mao et al. (2004) Science, 300, 213-217 describe in detail the techniques of burning the biomolecules off, both are incorporated herein by reference in their entireties. The annealing conditions can be chosen such that the nanoparticles remaining on the surface in the patterned areas are fused together.

Finally, it is clear that numerous variations and modifications may be made to method and apparatus described and illustrated herein, all falling within the scope of the invention as defined in the attached claims.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

1. A method of forming an integrated circuit layer material comprising:
depositing a layer of templates on a substrate, each said template including a first binding site having an affinity for the substrate, a second binding site having an affinity for a target integrated circuit material and a protecting material coupled to the second binding site via a labile linkage to prevent the binding site from binding to the target integrated circuit material;
exposing the template to an external stimulus to degrade the labile linkage;
removing the protecting material; and
binding the integrated circuit layer material to the second binding site.

2. (canceled)

3. The method of claim 1 wherein the template is a biomolecular template.

4. The method of claim 1 wherein the binding step comprises directly conjugating the integrated circuit layer material to the second binding site.

5. The method of claim 1 wherein the binding step comprises converting a precursor of the integrated circuit layer material to the integrated circuit layer material in a solution and nucleating the integrated circuit layer material on the template.

6-8. (canceled)

9. The method of claim 1 further comprising binding a seed material to the second binding site prior to the binding of the integrated circuit layer material.

10. The method of claim 9 wherein the seed material comprises nanoparticles and the integrated circuit layer material nucleates on the nanoparticles.

11. The method of claim 1 wherein the integrated circuit layer material is a metal, a metal oxide, a semiconductive material, an insulating material or a magnetic material.

12. A method comprising:
depositing a plurality of biomolecular templates on a substrate to form a template layer, each biomolecular template having a multifunctional biomolecule including a first binding site coupled to the substrate and a second binding site having an affinity for the target inorganic material, and a protecting group coupled to the multifunctional biomolecule via a labile linkage such that the second binding site is prevented from binding to the target inorganic material;
exposing, according to a selected pattern, a region of the template layer to an external stimulus;
deprotecting the second binding sites of the biomolecular template in the region subjected to the external stimulus by degrading the labile linkages thereof; and
binding the target inorganic material to the second binding sites in the region.

13. The method of claim 12 wherein the exposing step includes aligning, over the template layer, a mask having the selected pattern.

14. The method of claim 13 wherein the exposing step comprises irradiating the template layer with light and the labile linkage degrades in response to light.

15-22. (canceled)

23. The method of claim 12 wherein the multifunctional biomolecule is a peptide, antibody, block copolypeptide or amphiphilic lipopeptide.

24. The method of claim 12 wherein the target inorganic material includes a first nanoparticle.

25. (canceled)

26. The method of claim 24 further comprising nucleating a layer of integrated circuit material using the first nanoparticles as a seed material.

27-28. (canceled)

29. A biomolecular template comprising:
a multifunctional biomolecule including a first binding site having an affinity for a substrate and a second binding site having an affinity for a target inorganic material; and
a protecting group coupled to the multifunctional biomolecule via a labile linkage, the protecting group preventing the second binding site from binding to the target inorganic material.

30. The biomolecular template of claim 29 wherein the multifunctional biomolecule is a peptide, antibody, block copolypeptide or amphiphilic lipopeptide.

31-33. (canceled)

34. The biomolecular template of claim 29 wherein the labile linkage is degradable upon exposure to a light irradiation.

35. The biomolecular template of claim 34 wherein the protecting group is an ortho-nitrobenzyl derivative represented by Formula (I): 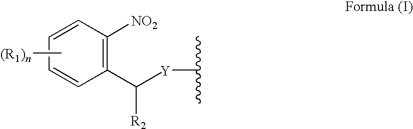
wherein:
each R₁ is the same or different and independently hydrogen, C₁₋₆ alkyl, —O—C₁₋₆ alkyl, NO₂, —CH₂COOH or —OH;
n is 0, 1, 2, 3 or 4;
R₂ is hydrogen, C₁₋₆ alkyl or —COOH; and
Y is a bond or —OC(O)—.

36-37. (canceled)

38. The biomolecular template of claim 29 wherein the target inorganic material is a seed material.

39. The biomolecular template of claim 38 wherein the seed material is a first nanoparticle.

40. The biomolecular template of claim 39 wherein the first nanoparticle is Au, Ni, Cu₁, Pd, Co, Pt, Ru₁, Ag, Cr₁, W, Mo, Co alloys or Ni alloys.

41. The biomolecular template of claim 39 wherein the first nanoparticle nucleates the growth of a layer of second target inorganic material.

42. The biomolecular template of claim 41 wherein the second inorganic material is Cu, Au₁, Ag, Ni, Pd, Co, Pt, Ru, Ag₁, Cr, W, Mo₁, Co alloys, Ni alloys, indium oxide, aluminum oxide, indium tin oxide, cobalt oxide, nickel oxide, copper oxide, zinc oxide, tin oxide, titanium oxide, tantalum oxide, hafnium oxide, niobium oxide, vanadium oxide or zirconium oxide.

43-89. (canceled)
